(19) United States
(12) Patent Application Publication
(10) Pub. No.: US 2025/0001010 A1
(43) Pub. Date: Jan. 2, 2025

(54) NRAS GENE KNOCKOUT FOR TREATMENT OF CANCER

(71) Applicant: Christiana Care Gene Editing Institute, Inc., Wilmington, DE (US)

(72) Inventors: Eric B. Kmiec, Middletown, DE (US); Sophia Masciarelli, Wilmington, DE (US); Brett Sansbury, Pilesgrove, NJ (US); Kelly H. Banas, Bear, DE (US)

(73) Assignee: Christiana Care Gene Editing Institute, Inc., Wilmington, DE (US)

(21) Appl. No.: 18/760,944

(22) Filed: Jul. 1, 2024

Related U.S. Application Data
(60) Provisional application No. 63/511,320, filed on Jun. 30, 2023.

(51) Int. Cl. A61K 48/00; A61K 45/06; A61P 35/00; C12N 9/22; C12N 15/11

(52) U.S. Cl. CPC A61K 48/005; A61K 45/06; A61P 35/00; C12N 9/22; C12N 15/111; C12N 2310/20

(57) ABSTRACT

The disclosure provides a guide RNA (gRNA) comprising a DNA-binding domain and a Clustered Regularly Interspaced Short Palindromic Repeat (CRISPR)-associated endonuclease protein-binding domain, wherein the DNA-binding domain is complementary to a target domain from an NRAS gene. The disclosure also provides nucleic acid sequence encoding the gRNA. The disclosure further provides a method of treating cancer in a subject comprising administering to the subject a therapeutically effective amount of a pharmaceutical composition comprising a CRISPR-associated endonuclease and a guide RNA that is complementary to a target domain from an NRAS gene in the subject. Methods of treating cancer comprising administering a pharmaceutical composition comprising: a nucleic acid sequence encoding a guide RNA that is complementary to a target domain from an NRAS gene in the subject; and a nucleic acid sequence encoding a CRISPR-associated endonuclease, are also provided.

### CROSS-REFERENCE TO RELATED APPLICATIONS

This application claims the benefit of U.S. Provisional Application 63/511,320, filed Jun. 30, 2023, which is incorporated herein, in its entirety, by reference.

### SUBMISSION OF SEQUENCE LISTING

The Sequence Listing associated with this application is filed in electronic format via Patent Center and hereby incorporated by reference into the specification in its entirety. The name of the text file containing the Sequence Listing is 13094901701 sequencelisting.xml. The size of the xml file is 11 KB, and the xml file was created on Aug. 29, 2024.

### FIELD

The present disclosure relates to compositions and methods for knocking out variant NRAS genes found in certain cancers to treat such cancers using Clustered Regularly Interspaced Short Palindromic Repeat (CRISPR)/endonuclease gene editing.

### BACKGROUND

Cancer is presently one of the leading causes of death in developed nations. A diagnosis of cancer traditionally involves serious health complications. Cancer can cause disfigurement, chronic or acute pain, lesions, organ failure, or even death. Commonly diagnosed cancers include lung cancer, pancreatic cancer, breast cancer, melanoma, lymphoma, carcinoma, sarcoma leukemia, endometrial cancer, colon and rectal cancer, prostate cancer, and bladder cancer. Traditionally, many cancers are treated with surgery, chemotherapy, radiation, or combinations thereof.

Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult. The lack of therapeutic options and increasing resistance to available drugs, creates a huge challenge in cancer therapy. Additionally, the high doses of chemotherapy required at advanced stage causes significant adverse side-effects, deteriorating the quality of life of the patients.

NRAS (also known as NS6, ALPS4, CMNS, and NCMS) encodes a GTPase, which is activated by a guanine nucleotide-exchange factor and inactivated by a GTPase activating protein. Mutations in this gene have been associated with somatic rectal cancer, follicular thyroid cancer, autoimmune lymphoproliferative syndrome, Noonan syndrome, and juvenile myelomonocytic leukemia.

NRAS interacts with the cell membrane and various effector proteins (e.g., Raf and RhoA), which carry out its signaling function through the cytoskeleton and effects on cell adhesion (Fotiadou et al., Mol. Cell. Biol. 27:6742-55 (2007)). Aberrant activation of the RAS pathway is a crucial event in many cancers and is frequently caused by point mutations of hotspot codons located within exon 2 (codons 12 and 13) and exon 3 (codon 61). The mutations disrupt intrinsic and RAS-GAP-mediated GTP hydrolysis, leading to constitutive activation and increased affinity of NRAS to the direct effectors, RAFs (ARAF, BRAF, and CRAFJ), RAS-like protein (RAL) guanine nucleotide exchange factors (GEFs), and PI3K (Eisfeld et al., Proc. Natl. Acad. Sci. USA 111:4179-84 (2014); Vu et al., Pharmacol. Res. 107:111-16 (2016)).

NRAS and BRAF both play a part in the mitogen-activated protein kinase (MAPK) pathway, which significantly contributes to melanoma development. In physiological conditions, the MAPK pathway is activated by growth factors binding to their surface receptor tyrosine kinase (RTK), and the signal is transmitted through NRAS (Cox et al., Nat. Rev. Drug Discov. 13:828-51 (2014)). However, despite more than three decades of effort by academia and industry, no effective anti-Ras inhibitors have reached the clinic, prompting a widely held perception that Ras oncoproteins are an “undruggable” cancer target.

NRAS mutations are found in melanoma, hematopoietic, and lymphoid tissue malignancies, and sometimes thyroid tumors (Dumaz et al., Cancers (Basel) 11:1133 (2019)).

Genes encoding members of the two canonical Ras effector families, BRAF and PIK3CA, are also frequently mutationally activated in human cancers (20% and 12%, respectively), supporting the importance of these pathways in driving cancer growth. The serine/threonine kinase B-raf is encoded by the BRAF gene. The most common BRAF alteration observed in human cancers including melanoma is the codon 600 valine to glutamate (V600E) mutation. BRAF V600 is located in the activation segment of the kinase domain close to T599 and S602 which on phosphorylation result in kinase activity. The V600E alteration may mimic the T599/S602 phosphorylation since it was shown that the V600E mutant B-raf has a higher kinase activity than wild type B-raf (Platz et al., Mol. Oncol. 1:395-405 (2008)).

BRAF mutations are present in approximately 8% of human tumors, but with huge variation in frequency depending on the malignancy. BRAF is commonly mutated in melanomas, papillary thyroid cancers, hairy cell leukemias, and idiopathic disorder Langerhans cell histiocytosis, and less frequently in colorectal cancers, lung adenocarcinomas, and hematopoietic and lymphoid tissue malignancies (Karoulia et al., Nat. Rev. Cancer 17:676-91 (2017)).

The clinical success of BRAF-targeted therapy and immunotherapy with high responders and long survivors suggests that short- and long-term disease control can be a reality for the unclearly defined subgroups of patients with melanoma. BRAF and NRAS co-mutations are not mutually exclusive; however, the sole finding of double-mutated cells in a resistant tumor is insufficient to determine follow-up therapy, and combinational therapy targeting different pathways will be necessary (Muñoz-Couselo et al., OncoTargets Ther. 10:3941-47 (2017)). Thus, despite prolonging patient survival, BRAF inhibitor treatment is rarely curative and is limited in most cases by the development of drug resistance and tumor relapse.

While there are many inhibitors directed to mutated BRAF, more specific and efficient NRAS-directed agents are still lacking. Resistance develops almost invariably during treatment with one inhibitor. In such setting, micropopulations of cells resistant to a drug gain growth advantage over others. This leads to the overgrowth of these cell populations and tumor resistance. Intratumoral heterogeneity is a driver of treatment resistance, causing phenotype switching and tumor plasticity (Vachtenheim et al., Life 11:424 (2021)).

Thus, a need exists for improved methods of treating cancer, in particular where BRAF inhibitor resistance has developed.

### SUMMARY

One aspect is for a method of reducing variant NRAS expression or activity in a cancer cell comprising introducing into the cancer cell (a) one or more nucleic acid sequences encoding one or more guide RNAs (gRNAs) that are complementary to one or more target sequences in a variant NRAS gene and (b) a nucleic acid sequence encoding a Clustered Regularly Interspaced Short Palindromic Repeat (CRISPR)-associated endonuclease, whereby the one or more gRNAs hybridize to the variant NRAS gene and the CRISPR-associated endonuclease cleaves the variant NRAS gene, and wherein NRAS expression or activity is reduced in the cancer cell relative to a cancer cell in which the one or more nucleic acid sequences encoding the one or more gRNAs and the nucleic acid sequence encoding the CRISPR-associated endonuclease are not introduced. In some embodiments, the one or more gRNAs comprise a trans-activated small RNA (tracrRNA) and/or a CRISPR RNA (crRNA). In some embodiments, the one or more gRNAs are one or more single guide RNAs. In some embodiments, the CRISPR-associated endonuclease is a class 2 CRISPR-associated endonuclease, and in some embodiments, the class 2 CRISPR-associated endonuclease is Cas12a or Cas9. In some embodiments, expression of one or more allele(s) of the variant NRAS gene is reduced in the cancer cell. In some embodiments, NRAS activity is reduced in the cancer cell. In some embodiments, NRAS expression or activity is not completely eliminated in the cancer cell. In some embodiments, NRAS expression or activity is completely eliminated in the cancer cell. In some embodiments, expression or activity of wild-type NRAS, in a non-cancer cell of a subject that contains the cancer cell is unaffected by the introduction of the one or more nucleic acid sequences of (a) and the nucleic acid sequence of (b). In some embodiments, the cancer cell is a lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor.

Another aspect is for a cancer cell comprising a mutated variant NRAS gene produced by the aforementioned method.

A further aspect is for a method of reducing variant NRAS expression or activity in a cancer cell comprising introducing into the cancer cell (a) one or more gRNAs that are complementary to one or more target sequences in the variant NRAS gene and (b) a CRISPR-associated endonuclease, whereby the one or more gRNAs hybridize to the variant NRAS gene and the CRISPR-associated endonuclease cleaves the variant NRAS gene, and wherein variant NRAS expression or activity is reduced in the cancer cell relative to a cancer cell in which the one or more gRNAs and the CRISPR-associated endonuclease are not introduced. In some embodiments, the one or more gRNAs comprise a tracrRNA and/or a crRNA. In some embodiments, the one or more gRNAs are one or more single guide RNAs. In some embodiments, the CRISPR-associated endonuclease is a class 2 CRISPR-associated endonuclease, and in some embodiments, the class 2 CRISPR-associated endonuclease is Cas12a or Cas9. In some embodiments, expression of one or more allele(s) of the variant NRAS gene is reduced in the cancer cell. In some embodiments, NRAS activity is reduced in the cancer cell. In some embodiments, NRAS expression or activity is not completely eliminated in the cancer cell. In some embodiments, NRAS expression or activity is completely eliminated in the cancer cell. In some embodiments, expression or activity of wild-type NRAS in a non-cancer cell of a subject that contains the cancer cell is unaffected by the introduction of the one or more nucleic acid sequences of (a) and the nucleic acid sequence of (b). In some embodiments, the cancer cell is a lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor.

An additional aspect is for a cancer cell comprising a mutated variant NRAS gene produced by the aforementioned method.

Another aspect is for a gRNA comprising a DNA-binding domain and a CRISPR-associated endonuclease protein-binding domain, wherein the DNA-binding domain is complementary to a target sequence in an NRAS gene. In some embodiments, the NRAS gene is a variant NRAS gene. In some embodiments, the gRNA comprises a tracrRNA and/or a crRNA. In some embodiments, the gRNA is a single guide RNA. In some embodiments, the gRNA comprises a crRNA.

A further aspect is for a pharmaceutical composition comprising the aforementioned gRNA. In some embodiments, the pharmaceutical composition further comprises a CRISPR-associated endonuclease; in some embodiments, the CRISPR-associated endonuclease is a class 2 CRISPR-associated endonuclease; and in some embodiments, the class 2 CRISPR-associated endonuclease is Cas12a or Cas9.

An additional aspect is for a ribonucleoprotein (RNP) complex comprising the aforementioned gRNA and a CRISPR-associated endonuclease. In some embodiments, the CRISPR-associated endonuclease is a class 2 CRISPR-associated endonuclease, and in some embodiments, the class 2 CRISPR-associated endonuclease is Cas12a and/or Cas9.

Another aspect is for a pharmaceutical composition comprising the aforementioned RNP complex.

A further aspect is for a nucleic acid sequence encoding the aforementioned gRNA, or a biologically active fragment thereof. In some embodiments, the biologically active fragment is a tracrRNA and/or a crRNA.

An additional aspect is for a vector comprising the aforementioned nucleic acid sequence. In some embodiments, the vector is an adeno-associated virus (AAV) vector. In some embodiments, the vector further comprises a nucleic acid sequence that encodes a CRISPR-associated endonuclease protein; in some embodiments, the CRISPR-associated endonuclease is a class 2 CRISPR-associated endonuclease; and in some embodiments, the class 2 CRISPR-associated endonuclease is Cas12a or Cas9.

Another aspect is for a pharmaceutical composition comprising the aforementioned nucleic acid sequence or the aforementioned vector.

A further aspect is for a pharmaceutical composition comprising the aforementioned nucleic acid sequence, further comprising a nucleic acid sequence that encodes a CRISPR-associated endonuclease protein. In some embodiments, the CRISPR-associated endonuclease is a class 2 CRISPR-associated endonuclease, and in some embodiments, the class 2 CRISPR-associated endonuclease is Cas12a or Cas9.

An additional aspect is for a method of treating cancer in a subject comprising administering to the subject a therapeutically effective amount of the aforementioned pharmaceutical composition. In some embodiments, expression or activity of wild-type NRAS in a non-cancer cell of the subject is unaffected by the administration of the pharmaceutical composition. In some embodiments, the cancer is resistant to one or more chemotherapeutic agents. In some embodiments, the method further comprises administering one or more chemotherapeutic agents to the subject; in some embodiments, the cancer is resistant to one or more BRAF inhibitors; and in some embodiments, the one or more BRAF inhibitors are vemurafenib, dabrafenib, encorafenib, sorafenib, tivatinib, ARQ736, ARQ680, AZ628, CEP-32496, GDC-0879, NMS-P186, NMS-P349, NMS-P383, NMS-P396, NMS-P730, PLX3603, PLX4720, PF-04880594, PLX4734, RAF265, R04987655, SB590885, BMS908662, WYE-130600, TAK632, MLN 2480, XL281, LUT001, LUT156, LUT192, LUT195, LUT197, or a combination thereof. In some embodiments, the variant NRAS comprises a Q61K mutation. In some embodiments, the pharmaceutical composition is administered in an amount sufficient to reduce proliferation of cells of the cancer relative to cancer cells that are not treated with the pharmaceutical composition. In some embodiments, the pharmaceutical composition is administered in an amount sufficient to reduce tumor growth relative to a tumor that is not treated with the pharmaceutical composition. In some embodiments, the pharmaceutical composition is administered in an amount sufficient to reduce proliferation of cells of the cancer relative to cancer cells that are treated with the at least one chemotherapeutic agent but are not treated with the pharmaceutical composition. In some embodiments, the pharmaceutical composition is administered in an amount sufficient to reduce tumor growth relative to a tumor that is treated with the at least one chemotherapeutic agent but is not treated with the pharmaceutical composition. In some embodiments, the subject is a human. In some embodiments, the cancer is a lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor.

Another aspect is for a CRISPR system for use as a medicament, the CRISPR system comprising (a) a gRNA that comprises a DNA-binding domain and a CRISPR-associated endonuclease protein-binding domain, wherein the DNA-binding domain is complementary to a target sequence in an NRAS gene, and (b) a CRISPR-associated endonuclease. In some embodiments, the NRAS gene is a variant NRAS gene. In some embodiments, the gRNA comprises a tracrRNA and/or a crRNA. In some embodiments, the gRNA is a single gRNA. In some embodiments, the gRNA comprises a crRNA.

A further aspect is for use of the aforementioned CRISPR system in treating cancer. In some embodiments, the cancer is resistant to one or more chemotherapeutic agent; in some embodiments, the cancer is resistant to one or more BRAF inhibitors; and in some embodiments, the one or more BRAF inhibitors are vemurafenib, dabrafenib, encorafenib, sorafenib, tivatinib, ARQ736, ARQ680, AZ628, CEP-32496, GDC-0879, NMS-P186, NMS-P349, NMS-P383, NMS-P396, NMS-P730, PLX3603, PLX4720, PF-04880594, PLX4734, RAF265, R04987655, SB590885, BMS908662, WYE-130600, TAK632, MLN 2480, XL281, LUT001, LUT156, LUT192, LUT195, LUT197, or a combination thereof. In some embodiments, the variant NRAS comprises a Q61K mutation. In some embodiments, the CRISPR system for use further comprises one or more chemotherapeutic agents, and in some embodiments, the one or more chemotherapeutic agents are selected from the group consisting of cisplatin, vinorelbine, carboplatin, and a combination thereof. In some embodiments, the CRISPR-associated endonuclease is a class 2 CRISPR-associated endonuclease, and in some embodiments, the class 2 CRISPR-associated endonuclease is Cas12a or Cas9.

An additional aspect is for an RNP complex for use as a medicament, the RNP complex comprising (a) a gRNA that comprises a DNA-binding domain and a CRISPR-associated endonuclease protein-binding domain, wherein the DNA-binding domain is complementary to a target sequence in an NRAS gene, and (b) a CRISPR-associated endonuclease. In some embodiments, the gRNA comprises a tracrRNA and/or a crRNA. In some embodiments, the gRNA is a single gRNA. In some embodiments, the gRNA comprises a crRNA. In some embodiments, the RNP complex further comprises one or more chemotherapeutic agents; in some embodiments, the cancer is resistant to one or more BRAF inhibitors; and in some embodiments, the one or more BRAF inhibitors are vemurafenib, dabrafenib, encorafenib, sorafenib, tivatinib, ARQ736, ARQ680, AZ628, CEP-32496, GDC-0879, NMS-P186, NMS-P349, NMS-P383, NMS-P396, NMS-P730, PLX3603, PLX4720, PF-04880594, PLX4734, RAF265, R04987655, SB590885, BMS908662, WYE-130600, TAK632, MLN 2480, XL281, LUT001, LUT156, LUT192, LUT195, LUT197, or a combination thereof. In some embodiments, the variant NRAS comprises a Q61K mutation. In some embodiments, the CRISPR-associated endonuclease is a class 2 CRISPR-associated endonuclease, and in some embodiments, the class 2 CRISPR-associated endonuclease is Cas12a or Cas9.

Another aspect is for use of the aforementioned RNP complex in treating cancer. In some embodiments, the cancer is resistant to one or more chemotherapeutic agents. In some embodiments, the RNP complex further comprises one or more chemotherapeutic agents. In some embodiments, the CRISPR-associated endonuclease is a class 2 CRISPR-associated endonuclease, and in some embodiments, the class 2 CRISPR-associated endonuclease is Cas12a or Cas9. In some embodiments, the cancer is a lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor.

A further aspect is for a method of reducing NRAS expression or activity in a cancer cell comprising introducing into the cancer cell (a) one or more nucleic acid sequences encoding one or more guide RNAs (gRNAs) that are complementary to one or more target sequences in a NRAS gene and (b) a nucleic acid sequence encoding a Clustered Regularly Interspaced Short Palindromic Repeat (CRISPR)-associated endonuclease, whereby the one or more gRNAs hybridize to the gene and the CRISPR-associated endonuclease cleaves the gene, and wherein NRAS expression or activity is reduced in the cancer cell relative to a cancer cell in which the one or more nucleic acid sequences encoding the one or more gRNAs and the nucleic acid sequence encoding the CRISPR-associated endonuclease are not introduced. In some embodiments, the cancer cell is a lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor.

An additional aspect is for a cancer cell comprising a mutated NRAS gene produced by the aforementioned method.

Another aspect is for a method of reducing NRAS expression or activity in a cancer cell comprising introducing into the cancer cell (a) one or more gRNAs that are complementary to one or more target sequences in the NRAS gene and (b) a CRISPR-associated endonuclease, whereby the one or more gRNAs hybridize to the NRAS gene and the CRISPR-associated endonuclease cleaves the NRAS gene, and wherein NRAS expression or activity is reduced in the cancer cell relative to a cancer cell in which the one or more gRNAs and the CRISPR-associated endonuclease are not introduced. In some embodiments, the cancer cell is a lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor.

A further aspect is for a cancer cell comprising a mutated NRAS gene produced by the aforementioned method.

An additional aspect is for a gRNA comprising a DNA-binding domain and a CRISPR-associated endonuclease protein-binding domain, wherein the DNA-binding domain is complementary to a target sequence in an NRAS gene.

Another aspect is for a pharmaceutical composition comprising the aforementioned gRNA.

A further aspect is for a ribonucleoprotein (RNP) complex comprising the aforementioned gRNA and a CRISPR-associated endonuclease.

An additional aspect is for a pharmaceutical composition comprising the aforementioned RNP complex.

Another aspect is for a nucleic acid sequence encoding the aforementioned gRNA, or a biologically active fragment thereof.

A further aspect is for a vector comprising the aforementioned nucleic acid sequence.

An additional aspect is for a pharmaceutical composition comprising the aforementioned nucleic acid sequence or the aforementioned vector.

Another aspect is for a pharmaceutical composition comprising the aforementioned nucleic acid sequence, further comprising a nucleic acid sequence that encodes a CRISPR-associated endonuclease protein.

A further aspect is for a method of treating cancer in a subject comprising administering to the subject a therapeutically effective amount of the aforementioned pharmaceutical composition. In some embodiments, the cancer is a lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor.

An additional aspect is for a CRISPR system for use as a medicament, the CRISPR system comprising (a) a gRNA that comprises a DNA-binding domain and a CRISPR-associated endonuclease protein-binding domain, wherein the DNA-binding domain is complementary to a target sequence in an NRAS gene, and (b) a CRISPR-associated endonuclease.

Another aspect is for an RNP complex for use as a medicament, the RNP complex comprising (a) a gRNA that comprises a DNA-binding domain and a CRISPR-associated endonuclease protein-binding domain, wherein the DNA-binding domain is complementary to a target sequence in an NRAS gene, and (b) a CRISPR-associated endonuclease.

A further aspect is for a method of reducing chemoresistance to a BRAF inhibitor in a cancer cell comprising introducing into the cancer cell (a) one or more nucleic acid sequences encoding one or more guide RNAs (gRNAs) that are complementary to one or more target sequences in a variant NRAS gene and (b) a nucleic acid sequence encoding a Clustered Regularly Interspaced Short Palindromic Repeat (CRISPR)-associated endonuclease, whereby the one or more gRNAs hybridize to the variant NRAS gene and the CRISPR-associated endonuclease cleaves the variant NRAS gene, and wherein chemoresistance to a BRAF inhibitor is reduced in the cancer cell relative to a cancer cell in which the one or more nucleic acid sequences encoding the one or more gRNAs and the nucleic acid sequence encoding the CRISPR-associated endonuclease are not introduced. In some embodiments, the one or more gRNAs comprise a trans-activated small RNA (tracrRNA) and/or a CRISPR RNA (crRNA). In some embodiments, the one or more gRNAs are one or more single guide RNAs. In some embodiments, the CRISPR-associated endonuclease is a class 2 CRISPR-associated endonuclease; and in some embodiments, the class 2 CRISPR-associated endonuclease is Cas12a. In some embodiments, the one or more BRAF inhibitors are vemurafenib, dabrafenib, encorafenib, sorafenib, tivatinib, ARQ736, ARQ680, AZ628, CEP-32496, GDC-0879, NMS-P186, NMS-P349, NMS-P383, NMS-P396, NMS-P730, PLX3603, PLX4720, PF-04880594, PLX4734, RAF265, R04987655, SB590885, BMS908662, WYE-130600, TAK632, MLN 2480, XL281, LUT001, LUT156, LUT192, LUT195, LUT197, or a combination thereof. In some embodiments, the cancer comprises a variant NRAS having a Q61K mutation.

Other objects and advantages will become apparent to those skilled in the art upon reference to the detailed description that hereinafter follows.

### BRIEF DESCRIPTION OF THE DRAWINGS

FIG. 1. Mechanism of BRAFi resistance through an acquired NRAS 061K mutation. The Ras-Raf-MEK-ERK pathway is shown as influenced by BRAF/NRAS mutational status, noted as wild-type (WT) or mutated (V600E or Q61K). The influence of BRAFi treatment on BRAF^{V600E}/NRAS^{WT} is shown inhibiting the MAPK pathway while BRAF^{V600E}/NRAS^{Q61K} status shows the mechanism of inhibition escape and MAPK overexpression.

FIG. 2A. Mechanism of BRAFi resistance through an acquired NRAS 061K mutation. The Ras-Raf-MEK-ERK pathway is shown as influenced by BRAF/NRAS mutational status, noted as wild-type (WT) or mutated (V600E or Q61K). The influence of BRAFi treatment on BRAF^{V600E}/NRAS^{WT} is shown inhibiting the MAPK pathway while BRAF^{V600E}/NRAS^{Q61K} status shows the mechanism of inhibition escape and MAPK overexpression. Restored sensitivity to BRAFi treatment is shown after NRAS Q61K MT-selective editing. FIG. 2B. BRAFi dosing curves for Dabrafenib and Vemurafenib. Isogenic and parental A375 cells were dosed with concentrations ranging from 0 nM to 2000 nM. Cell viability was read after 72 hours.

FIG. 3. NRAS Q61K mutation site and selective targeting. FIG. 3A. The NRAS Q61K mutation site is shown and a representation of the allelic contributions of BRAF^{V600E}/NRAS^{Q61K} A375 cell line is shown here, with 66% WT and 33% MT contributions. FIG. 3B. Two NRAS Q61K-selective CRISPR target sites are illustrated here, with the Cas12a (nPAM) PAM site on the 5′ end relative to the spacer and the SaCas9 (sMT) PAM site on the 3′ end relative to the spacer. FIG. 3C. Genomic analysis of Q61K-selective Cas12a nPAM targeting. NGS analysis is shown for Q61K-selective Cas12a nPAM targeting reaction. The percentage of Q61K contribution remaining after targeting are shown as bold text. FIG. 3D. A bar graph is shown representing the percentage of sequencing contributions after editing for Q61 wild-type NRAS, Q61K mutated NRAS, and indels.

FIG. 4. Genomic analysis of WT- and MT-selective SaCas9 sMT targeting. FIG. 4A. Selective (SaCas9 sMT and Cas12a nPAM) and non-selective (SpCas9) targets are shown at the Q61K mutation site. FIG. 4B. NGS sequencing is shown of selective (SaCas9 sMT and Cas12a nPAM) and non-selective (SpCas9) targeted populations. The percentage of Q61K contribution remaining after targeting are shown as bold text. FIG. 4C. A bar graph is shown representing the percentage of sequencing contributions after editing for Q61 wild-type NRAS, Q61K mutated NRAS, and indels.

FIG. 5. BRAFi sensitivity after 061K-selective targeting. Cell viability data in response to BRAFi treatment with FIG. 5A Dabrafenib and FIG. 5B Vemurafenib is shown for selective and non-selective targeting reactions, compared with Parental (BRAFi-sensitive) and Isogenic (BRAFi-resistant) A375 cells and transfection controls.

FIG. 6 Characterization of prominent editing outcomes for selective and non-selective targeting. A characterization of the NRAS gene resulting from prominent editing outcomes after targeting with FIG. 6A SaCas9 sMT, FIG. 6B Cas12a nPAM and FIG. 6C SpCas9 are shown. Deletions can be seen as gaps in sequence, with inserted bases stop codons also shown.

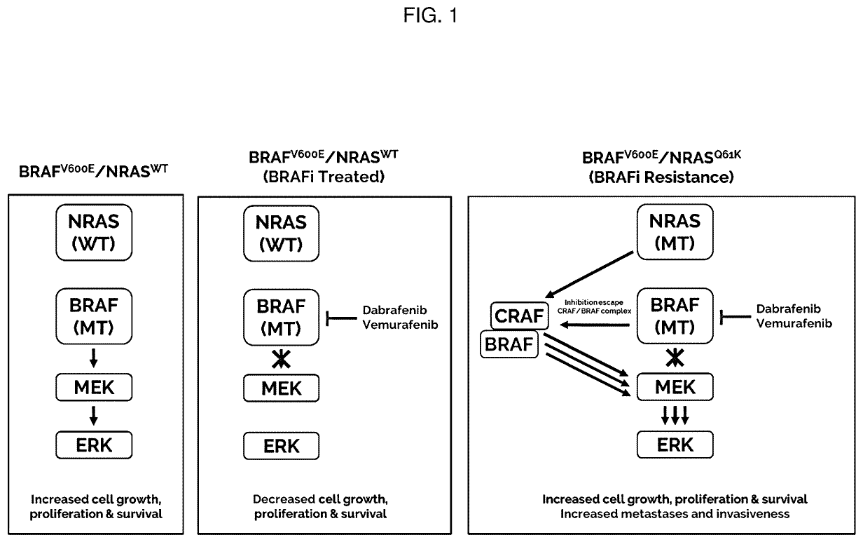

### DETAILED DESCRIPTION

### Definitions

Applicant specifically incorporates the entire contents of all cited references in this disclosure. Further, when an amount, concentration, or other value or parameter is given as either a range or a list of upper values and lower values, this is to be understood as specifically disclosing all ranges formed from any pair of any upper range limit or value and any lower range limit or value, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

The indefinite articles “a” and “an”, as used herein in the specification and in the claims, unless clearly indicated to the contrary, should be understood to mean “at least one”.

The phrase “and/or”, as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, “either”, “one of”, “only one of”, “exactly one of”. “Consisting essentially of”, when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

A “Clustered Regularly Interspaced Short Palindromic Repeat (CRISPR)-associated endonuclease protein-binding domain” or “Cas binding domain” refers to a nucleic acid element or domain within a nucleic acid sequence or polynucleotide sequence that, in an effective amount, will bind or have an affinity for one or a plurality of CRISPR-associated endonuclease (or functional fragments thereof). In some embodiments, in the presence of the one or a plurality of proteins (or functional fragments thereof) and a target sequence, the one or plurality of proteins and the nucleic acid element forms a biologically active CRISPR complex and/or can be enzymatically active on a target sequence. In some embodiments, the CRISPR-associated endonuclease is a class 1 or class 2 CRISPR-associated endonuclease, and in some embodiments, a Cas9 or Cas12a endonuclease. The Cas9 endonuclease can have a nucleotide sequence identical to the wild type Streptococcus pyogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Such species include: Acidovorax avenae, Actinobacillus pleuropneumoniae, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., and Verminephrobacter eiseniae (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas9 endonucleases). In some embodiments, the CRISPR-associated endonuclease can be a Cas12a nuclease. The Cas12a nuclease can have a nucleotide sequence identical to a wild type Prevotella or Francisella sequence (or functional fragments or variants of any of the aforementioned sequences that have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to any of the aforementioned Cas12 endonucleases).

In some embodiments, the terms “(CRISPR)-associated endonuclease protein-binding domain” or “Cas binding domain” refer to a nucleic acid element or domain (e.g. and RNA element or domain) within a nucleic acid sequence that, in an effective amount, will bind to or have an affinity for one or a plurality of CRISPR-associated endonucleases (or functional fragments or variants thereof that are at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to a CRISPR-associated endonuclease). In some embodiments, the Cas binding domain consists of at least or no more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially associates or binds to a biologically active CRISPR-associated endonuclease at a concentration and within a microenvironment suitable for CRISPR system formation.

The “Clustered Regularly Interspaced Short Palindromic Repeats (CRISPR)-CRISPR associated (Cas) (CRISPR-Cas) system guide RNA” or “CRISPR-Cas system guide RNA” may comprise a transcription terminator domain. The term “transcription terminator domain” refers to a nucleic acid element or domain within a nucleic acid sequence (or polynucleotide sequence) that, in an effective amount, prevents bacterial transcription when the CRISPR complex is in a bacterial species and/or creates a secondary structure that stabilizes the association of the nucleic acid sequence to one or a plurality of Cas proteins (or functional fragments thereof) such that, in the presence of the one or a plurality of proteins (or functional fragments thereof), the one or plurality of Cas proteins and the nucleic acid element forms a biologically active CRISPR complex and/or can be enzymatically active on a target sequence in the presence of such a target sequence and a DNA-binding domain. In some embodiments, the transcription terminator domain consists of at least or no more than about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, or 250 nucleotides and comprises at least one sequence that is capable of forming a hairpin or duplex that partially drives association of the nucleic acid sequence (sgRNA, crRNA with tracrRNA, or other nucleic acid sequence) to a biologically active CRISPR complex at a concentration and microenvironment suitable for CRISPR complex formation.

The term “DNA-binding domain” refers to a nucleic acid element or domain within a nucleic acid sequence (e.g. a guide RNA) that is complementary to NRAS. In some embodiments, the DNA-binding domain will bind or have an affinity for an NRAS gene such that, in the presence of a biologically active CRISPR complex, one or plurality of Cas proteins can be enzymatically active on the target sequence. In some embodiments, the DNA binding domain comprises at least one sequence that is capable of forming Watson Crick basepairs with a target sequence as part of a biologically active CRISPR system at a concentration and microenvironment suitable for CRISPR system formation.

“CRISPR system” refers collectively to transcripts or synthetically produced transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a nucleic acid sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, the target sequence is a DNA polynucleotide and is referred to a DNA target sequence. In some embodiments, a target sequence comprises at least three nucleic acid sequences that are recognized by a Cas-protein when the Cas protein is associated with a CRISPR complex or system which comprises at least one sgRNA or one tracrRNA/crRNA duplex at a concentration and within an microenvironment suitable for association of such a system. In some embodiments, the target DNA comprises at least one or more proto-spacer adjacent motifs which sequences are known in the art and are dependent upon the Cas protein system being used in conjunction with the sgRNA or crRNA/tracrRNAs employed by this work. In some embodiments, the target DNA comprises NNG, where G is an guanine and N is any naturally occurring nucleic acid. In some embodiments the target DNA comprises any one or combination of NNG, NNA, GAA, NNAGAAW and NGGNG, where G is an guanine, A is adenine, and N is any naturally occurring nucleic acid

In some embodiments, a target sequence is located in the nucleus or cytoplasm of a cell.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional (bind the Cas protein or functional fragment thereof). In some embodiments, the tracr sequence has at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of sequence complementarity along the length of the tracr mate sequence when optimally aligned. In some embodiments, one or more vectors driving expression of one or more elements of a CRISPR system are introduced into a host cell such that the presence and/or expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. With at least some of the modification contemplated by this disclosure, in some embodiments, the guide sequence or RNA or DNA sequences that form a CRISPR complex are at least partially synthetic. The CRISPR system elements that are combined in a single vector may be arranged in any suitable orientation, such as one element located 5′ with respect to (“upstream” of) or 3′ with respect to (“downstream” of) a second element. In some embodiments, the disclosure relates to a composition comprising a chemically synthesized guide sequence. In some embodiments, the chemically synthesized guide sequence is used in conjunction with a vector comprising a coding sequence that encodes a CRISPR enzyme, such as a class 2 Cas9 or Cas12a protein. In some embodiments, the chemically synthesized guide sequence is used in conjunction with one or more vectors, wherein each vector comprises a coding sequence that encodes a CRISPR enzyme, such as a class 2 Cas9 or Cas12a protein. The coding sequence of one element may be located on the same or opposite strand of the coding sequence of a second element, and oriented in the same or opposite direction. In some embodiments, a single promoter drives expression of a transcript encoding a CRISPR enzyme and one or more additional (second, third, fourth, etc.) guide sequences, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, one or more additional guide sequence, tracr mate sequence, and tracr sequence are each a component of different nucleic acid sequences. For instance, in the case of a tracr and tracr mate sequences and in some embodiments, the disclosure relates to a composition comprising at least a first and second nucleic acid sequence, wherein the first nucleic acid sequence comprises a tracr sequence and the second nucleic acid sequence comprises a tracr mate sequence, wherein the first nucleic acid sequence is at least partially complementary to the second nucleic acid sequence such that the first and second nucleic acid for a duplex and wherein the first nucleic acid and the second nucleic acid either individually or collectively comprise a DNA-targeting domain, a Cas protein binding domain, and a transcription terminator domain. In some embodiments, the CRISPR enzyme, one or more additional guide sequence, tracr mate sequence, and tracr sequence are operably linked to and expressed from the same promoter. In some embodiments, the disclosure relates to compositions comprising any one or combination of the disclosed domains on one guide sequence or two separate tracrRNA/crRNA sequences with or without any of the disclosed modifications. Any methods disclosed herein also relate to the use of tracrRNA/crRNA sequence interchangeably with the use of a guide sequence, such that a composition may comprise a single synthetic guide sequence and/or a synthetic tracrRNA/crRNA with any one or combination of modified domains disclosed herein.

In some embodiments, a guide RNA can be a short, synthetic, chimeric tracrRNA/crRNA (a “single-guide RNA” or “sgRNA”). A guide RNA may also comprise two short, synthetic tracrRNA/crRNAs (a “dual-guide RNA” or ‘dgRNA”).

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features.

As used herein, “cancer” refers to all types of cancer or neoplasm or malignant tumors found in humans, including, but not limited to: leukemias, lymphomas, melanomas, carcinomas and sarcomas. As used herein, the terms or language “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but also cancer stem cells, as well as cancer progenitor cells or any cell derived from a cancer cell ancestor. This includes metastasized cancer cells, and in vitro cultures and cell lines derived from cancer cells. In certain embodiments, the cancer is a blood tumor (i.e., a non-solid tumor). In some embodiments, the cancer is lymphoid neoplasm diffuse large B-cell lymphoma, cholangiocarcinoma, uterine carcinosarcoma, kidney chromophobe, uveal melanoma, mesothelioma, adrenocortical carcinoma, thymoma, acute myeloid leukemia, testicular germ cell tumor, rectum adenocarcinoma, pancreatic adenocarcinoma, phenochromocytoma and paraganglioma, esophageal carcinoma, sarcoma, kidney renal papillary cell carcinoma, cervical squamous cell carcinoma and endocervical adenocarcinoma, kidney renal clear cell carcinoma, liver hepatocellular carcinoma, glioblastoma multiforme, bladder urothelial carcinoma, colon adenocarcinoma, stomach adenocarcinoma, ovarian serous cystadenocarcinoma, skin cutaneous melanoma, prostate adenocarcinoma, thyroid carcinoma, lung squamous cell carcinoma, head and neck squamous cell carcinoma, brain lower grade glioma, uterine corpus endometrial carcinoma, lung adenocarcinoma, multiple myeloma, breast invasive carcinoma, acute lymphoblastic leukemia, acute myeloid leukemia, adrenocortical carcinoma, Kaposi sarcoma, AIDS-related lymphoma, primary CNS lymphoma, anal cancer, astrocytoma, atypical teratoid/rhabdoid tumor, bile duct cancer, bladder cancer, bone cancer, brain tumor, breast cancer, bronchial tumors, carcinoid tumor, carcinoma of unknown primary, cardiac tumor, medulloblastoma, germ cell tumor, cervical cancer, chordoma, chronic lymphocytic leukemia, chronic myelogenous leukemia, chronic myeloproliferative neoplasm, colorectal cancer, craniopharyngioma, embryonal tumor, endometrial cancer, ependymoma, esophageal cancer, esthesioneuroblastoma, Ewing sarcoma, extracranial germ cell tumor, extragonadal germ cell tumor, intraocular melanoma, retinoblastoma, fallopian tube cancer, fibrous histiocytoma of bone, osteosarcoma, gallbladder cancer, gastric cancer, gastrointestinal carcinoid tumor, gastrointestinal stromal tumor, CNS germ cell tumor, ovarian germ cell tumor, testicular cancer, gestational trophoblastic disease, hairy cell leukemia, head and neck cancer, hepatocellular cancer, Langerhans cell histiocytosis, Hodgkin lymphoma, hypopharyngeal cancer, islet cell tumor, kidney cancer, laryngeal cancer, leukemia, lip and oral cavity cancer, lung cancer (non-small cell, small cell, pleuropulmonary blastoma, tracheobronchial tumor), lymphoma, male breast cancer, malignant fibrous histiocytoma of bone, melanoma, Merkel cell carcinoma, malignant mesothelioma, metastatic cancer, metastatic squamous cell neck cancer with occult primary, midline tract carcinoma, mouth cancer, multiple endocrine neoplasia, plasma cell neoplasm, mycosis fungoides, myelodysplastic syndrome, myelodysplastic neoplasm, nasal cavity and paranasal sinus cancer, nasopharyngeal cancer, neuroblastoma, non-Hodgkin lymphoma, oropharyngeal cancer, ovarian cancer, pancreatic cancer, papillomatosis, paraganglioma, parathyroid cancer, pharyngeal cancer, pheochromocytoma, pituitary tumor, pleuropulmonary blastoma, primary peritoneal cancer, prostate cancer, rectal cancer, rhabdomyosarcoma, salivary gland cancer, Sézary syndrome, skin cancer, small intestine cancer, soft tissue sarcoma, testicular cancer, thymoma, thymic carcinoma, thyroid cancer, transitional cell cancer of the renal pelvis and ureter, urethral cancer, endometrial uterine cancer, uterine sarcoma, vaginal cancer, vascular tumor, vulvar cancer, or Wilms tumor (see, e.g., Kerins et al., Sci. Rep. 8:12846 (2018)).

In certain embodiments, the cancer is a solid tumor. A “solid tumor” is a tumor that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. The tumor does not need to have measurable dimensions.

Specific criteria for the staging of cancer are dependent on the specific cancer type based on tumor size, histological characteristics, tumor markers, and other criteria known by those of skill in the art. Generally, cancer stages can be described as follows:
Stage 0—Carcinoma in situ
Stage I, Stage II, and Stage III—Higher numbers indicate more extensive disease: Larger tumor size and/or spread of the cancer beyond the organ in which it first developed to nearby lymph nodes and/or tissues or organs adjacent to the location of the primary tumor
Stage IV—The cancer has spread to distant tissues or organs

As used herein, a “variant”, “mutant”, or “mutated” polynucleotide contains at least one polynucleotide sequence alteration as compared to the polynucleotide sequence of the corresponding wild-type or parent polynucleotide. Mutations may be natural, deliberate, or accidental. Mutations include substitutions, deletions, and insertions.

As used herein, the terms “treat,” “treating” or “treatment” refer to an action to obtain a beneficial or desired clinical result including, but not limited to, alleviation or amelioration of one or more signs or symptoms of a disease or condition (e.g., regression, partial or complete), diminishing the extent of disease, stability (i.e., not worsening, achieving stable disease) of the state of disease, amelioration or palliation of the disease state, diminishing rate of or time to progression, and remission (whether partial or total). “Treatment” of a cancer can also mean prolonging survival as compared to expected survival in the absence of treatment. Treatment need not be curative. In certain embodiments, treatment includes one or more of a decrease in pain or an increase in the quality of life (QOL) as judged by a qualified individual, e.g., a treating physician, e.g., using accepted assessment tools of pain and QOL. In certain embodiments, a decrease in pain or an increase in the QOL as judged by a qualified individual, e.g., a treating physician, e.g., using accepted assessment tools of pain and QOL is not considered to be a “treatment” of the cancer.

“Chemotherapeutic agent” refers to a drug used for the treatment of cancer. Chemotherapeutic agents include, but are not limited to, small molecules, hormones and hormone analogs, and biologics (e.g., antibodies, peptide drugs, nucleic acid drugs). In certain embodiments, chemotherapy does not include hormones and hormone analogs.

A “cancer that is resistant to one or more chemotherapeutic agents” is a cancer that does not respond, or ceases to respond to treatment with a chemotherapeutic regimen, i.e., does not achieve at least stable disease (i.e., stable disease, partial response, or complete response) in the target lesion either during or after completion of the chemotherapeutic regimen. Resistance to one or more chemotherapeutic agents results in, e.g., tumor growth, increased tumor burden, and/or tumor metastasis.

A “therapeutically effective amount” is that amount sufficient, at dosages and for periods of time necessary, to achieve a desired therapeutic result, such as for treatment of a disease (e.g. cancer), condition, or disorder, and/or pharmacokinetic or pharmacodynamic effect of the treatment in a subject. A therapeutically effective amount can be administered in one or more administrations. The therapeutically effective amount may vary according to factors such as the disease state, age, sex, and weight of the subject.

### CRISPR/Endonucleases

CRISPR/endonuclease (e.g., CRISPR/Cas9) systems are known in the art and are described, for example, in U.S. Pat. No. 9,925,248, which is incorporated by reference herein in its entirety. CRISPR-directed gene editing can identify and execute DNA cleavage at specific sites within the chromosome at a surprisingly high efficiency and precision. The natural activity of CRISPR/Cas9 is to disable a viral genome infecting a bacterial cell. Subsequent genetic reengineering of CRISPR/Cas function in human cells presents the possibility of disabling human genes at a significant frequency.

In bacteria, the CRISPR/Cas loci encode RNA-guided adaptive immune systems against mobile genetic elements (viruses, transposable elements and conjugative plasmids). Three types (I-III) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA) containing a DNA binding region (spacer) which is complementary to the target gene. The CRISPR-associated endonuclease, Cas9, belongs to the type II CRISPR/Cas system and has strong endonuclease activity to cut target DNA. Cas9 is guided by a mature crRNA that contains about 20 base pairs (bp) of unique target sequence (called a spacer) and a trans-activated small RNA (tracrRNA) that serves as a guide for ribonuclease III-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3rd nucleotide from PAM).

The compositions described herein can include a nucleic acid encoding a CRISPR-associated endonuclease. The CRISPR-associated endonuclease can be, e.g., a class 1 CRISPR-associated endonuclease or a class 2 CRISPR-associated endonuclease. Class 1 CRISPR-associated endonucleases include type I, type III, and type IV CRISPR-Cas systems, which have effector molecules that comprise multiple subunits. For class 1 CRISPR-associated endonucleases, effector molecules can include, in some embodiments, Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8e, and Cas6; Cas8f and Cas6f; Cas6f; Cas8-like (Csf1); SS (Cas11) and Cas8-like (Csf1); or SS (Cas11) and Cas10. Class 1 CRISPR-associated endonucleases also be associated with, in some embodiments, target cleavage molecules, which can be Cas3 (type I) or Cas10 (type III) and spacer acquisition molecules such as, e.g., Cas1, Cas2, and/or Cas4. See, e.g., Koonin et al., Curr. Opin. Microbiol. 37:67-78 (2017); Strich & Chertow, J. Clin. Microbiol. 57:1307-18 (2019).

Class 2 CRISPR-associated endonucleases include type I, type V, and type VI CRISPR-Cas systems, which have a single effector molecule. For class 2 CRISPR-associated endonucleases, effector molecules can include, in some embodiments, Cas9, Cas12a (cpf1), Cas12b1 (c2c1), Cas12a2, Cas12b2, Cas12c (c2c3), Cas12d (CasY), Cas12e (CasX), Cas12f1 (Cas14a), Cas12f2 (Cas14b), Cas12f3 (Cas14c), Cas12g, Cas12h, Cas12i, Cas12j (Casϕ), Cas12k (c2c5), Cas13a (c2c2), Cas13b1 (c2c6), Cas13b2 (c2c6), Cas13bt, Cas13c (c2c7), Cas13ct, Cas13d, Cas13X, Cas13Y, c2c4, c2c8, c2c9, and/or c2c10. See, e.g., Koonin et al., Curr. Opin. Microbiol. 37:67-78 (2017); Strich & Chertow, J. Clin. Microbiol. 57:1307-18 (2019); Makarova et al., Nat. Rev. Microbiol. 18:67-83 (2020); Pausch et al., Science 369:333-37 (2020); Tong et al., Cell Dev. Biol. 8:622103 (2021); Xu et al., Nat. Meth. 18:499-506 (2021); Kannan et al., Nat. Biotechnol. 40:194-97 (2022); Liu et al., Mol. Cell 82:333-47 (2022).

In some embodiments, the CRISPR-associated endonuclease can be a Cas9 nuclease. The Cas9 nuclease can have a nucleotide sequence identical to the wild type Streptococcus pyogenes sequence. In some embodiments, the CRISPR-associated endonuclease can be a sequence from other species, for example other Streptococcus species, such as agalactiae, anginosis, canis, castoreus, constella, constellatus, denstasini, devriesei, dysgalactiae, equi, equinus, gallolyticus, infantarius, iniae, lutetiensis, macacae, massiliensis, mitis, mutans, ovis, parasanguinis, parauberis, phocae, pseudoporcinus, plurextorum, ratti, sanguinis, sobrinus, suis, thermophilus, or tigurinus; Pseudomonas aeruginosa, Escherichia coli, or other sequenced bacteria genomes and archaea; or other prokaryotic microorganisms. Such species include: Acidaminoccus sp., Acidovorax avenae, Actinobacillus pleuropneumoniae, Actinobacillus succinogenes, Actinobacillus suis, Actinomyces sp., Alicyclobacillus acidiphilus, Alicyclobacillus acidoterrestris, Aminomonas paucivorans, Bacillus cereus, Bacillus hisahsii, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Cycliphilus denitrificans, Dinoroseobacter shibae, Dolosigranulum pigrum, Enterococcus durans, Enterococcus faecalis, Enterococcus faecium, Enterococcus hirae, Enterococcus italicus, Enterococcus mundtii, Enterococcus phoeniculicola, Enterococcus villorum, Eubacterium dolichum, Francisella novicida, Gammaproteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lachnospiraceae bacterium, Lactobacillus apodemi, Lactobacillus animalis, Lactobacillus crispatus, Leptotrichia shahii, Listeria innocua, Listeria selligeri, Listeria ivanovii, Listeria monocytogenes, Listeriaceae bacterium, Methylocystis sp., Methylosinus trichosporium, Mobiluncus mulieris, Moraxella bovoculi, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Prevotella bryantii, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus auricularis, Staphylococcus lugdunensis, Streptococcus canis, Streptococcus sp., Subdoligranulum sp., Sulfuricurvum sp., Tistrella mobilis, Treponema sp., and Verminephrobacter eiseniae.

Alternatively, the wild type Streptococcus pyogenes Cas9 sequence can be modified. The nucleic acid sequence can be codon optimized for efficient expression in mammalian cells, e.g., human cells. A Cas9 nuclease sequence codon optimized for expression in human cells sequence can be for example, the Cas9 nuclease sequence encoded by any of the expression vectors listed in Genbank accession numbers NZ_LS483338.1 GI:69900935, KM099231.1 GI:669193757; KM099232.1 GI:669193761; or KM099233.1 GI:669193765. Alternatively, the Cas9 nuclease sequence can be, for example, the sequence contained within a commercially available vector such as pX458, pX330 or pX260 from Addgene (Cambridge, Mass.). In some embodiments, the Cas9 endonuclease can have an amino acid sequence that is a variant or a fragment of any of the Cas9 endonuclease sequences of Genbank accession numbers NZ_LS483338.1 GI:69900935, KM099231.1 GI:669193757; KM099232.1 GI:669193761; or KM099233.1 GI:669193765 or Cas9 amino acid sequence of pX458, pX330 or pX260 (Addgene, Cambridge, Mass.). The Cas9 nucleotide sequence can be modified to encode biologically active variants of Cas9, and these variants can have or can include, for example, an amino acid sequence that differs from a wild type Cas9 by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas9 polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas9 polypeptide. See, e.g., US2019/0032036, US2023/0075913, US2023/0031899, US2023/0021641, US2022/0307001, US2022/0235340, US2022/0204954, US2022/0154158, US2022/0154157, US2021/0301269, US2021/0284978, US2021/0261932, US2021/0163907, US2021/0147861, US2020/0332271, US2020/0318086, US20200299657, US2020/0277586, US2020/0199552; each of which incorporated by reference herein in its entirety.

In some embodiments, the CRISPR-associated endonuclease can be a Cas12a nuclease. The Cas12a nuclease can have a nucleotide sequence identical to a wild type Prevotella or Francisella sequence. Alternatively, a wild type Prevotella or Francisella Cas12a sequence can be modified. The nucleic acid sequence can be codon optimized for efficient expression in mammalian cells, e.g., human cells. A Cas12a nuclease sequence codon optimized for expression in human cells sequence can be for example, the Cas12a nuclease sequence encoded by any of the expression vectors listed in Genbank accession numbers NZ_CP010070.1 GI:24818655, MF193599.1 GI:1214941796, KY985374.1 GI:1242863785, KY985375.1 GI:1242863787, or KY985376.1 GI:1242863789. Alternatively, the Cas12a nuclease sequence can be, for example, the sequence contained within a commercially available vector such as pAs-Cpf1 or pLb-Cpf1 from Addgene (Cambridge, Mass.). In some embodiments, the Cas12a endonuclease can have an amino acid sequence that is a variant or a fragment of any of the Cas12a endonuclease sequences of Genbank accession numbers NZ_CP010070.1 GI:24818655, MF193599.1 GI:1214941796, KY985374.1 GI:1242863785, KY985375.1 GI:1242863787, or KY985376.1 GI: 1242863789 or Cas12a amino acid sequence of pAs-Cpf1 or pLb-Cpf1 (Addgene, Cambridge, Mass.). The Cas12a nucleotide sequence can be modified to encode biologically active variants of Cas12a, and these variants can have or can include, for example, an amino acid sequence that differs from a wild type Cas12a by virtue of containing one or more mutations (e.g., an addition, deletion, or substitution mutation or a combination of such mutations). One or more of the substitution mutations can be a substitution (e.g., a conservative amino acid substitution). For example, a biologically active variant of a Cas12a polypeptide can have an amino acid sequence with at least or about 50% sequence identity (e.g., at least or about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity) to a wild type Cas12a polypeptide. See, e.g., US2019/0233814, US2019/0264186, US2023/0040148, US2021/0348144, US2021/0309701, US2021/0230567, US2021/0155911, US2020/0263190, US2020/0216825, US2021/0115421, US2021/0079366, US2020/0255861, US2019/0010481; each of which incorporated by reference herein in its entirety.

The compositions described herein may also include sequence encoding a gRNA comprising a DNA-binding domain that is complementary to a target domain from an NRAS gene, and a CRISPR-associated endonuclease protein-binding domain. In some embodiments, the gRNA comprises a DNA-binding domain that is complementary to a target domain from a variant NRAS gene that is found only in cancer cells and not in wild-type NRAS gene in normal (i.e., non-cancerous) cells. The guide RNA sequence can be a sense or anti-sense sequence. The sequence of the PAM can vary depending upon the specificity requirements of the CRISPR endonuclease used. In the CRISPR-Cas system derived from S. pyogenes, the target DNA typically immediately precedes a 5′-NGG or 5′-NAG proto-spacer adjacent motif (PAM). Thus, for example, the S. pyogenes Cas9, the PAM sequence can be AGG, TGG, CGG, GGG, AAG, TAG, CAG, or GAG. Other Cas9 orthologs and other Cas proteins (e.g., Cas12a's PAM site is TTTV) may have different PAM specificities. The specific sequence of the guide RNA may vary, but, regardless of the sequence, useful guide RNA sequences will be those that minimize off-target effects while achieving high efficiency. In some embodiments, the guide RNA sequence achieves complete ablation of the NRAS gene. In some embodiments, the guide RNA sequence achieves complete ablation of a variant NRAS gene without affecting expression or activity of a wild-type NRAS gene.

In some embodiments, the DNA-binding domain varies in length from about 16 to about 55 nucleotides, for example, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides. In some embodiments, the Cas protein-binding domain is from about 30 to about 55 nucleotides in length, for example, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, or about 55 nucleotides.

In some embodiments, the compositions comprise one or more nucleic acid (e.g., DNA or RNA) sequences encoding the guide RNA and the CRISPR endonuclease. When the compositions are administered as a nucleic acid or are contained within an expression vector, the CRISPR endonuclease can be encoded by the same nucleic acid or vector as the guide RNA sequence. In some embodiments, the CRISPR endonuclease can be encoded in a physically separate nucleic acid from the guide RNA sequence or in a separate vector. The nucleic acid sequence encoding the guide RNA may comprise a DNA binding domain, a Cas protein binding domain, and a transcription terminator domain.

The nucleic acid encoding the guide RNA and/or the CRISPR endonuclease may be an isolated nucleic acid. An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein. PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids also can be obtained by mutagenesis of, e.g., a naturally occurring portion of a Cas9-encoding DNA (in accordance with, for example, the formula above).

Recombinant constructs are also provided herein and can be used to transform cells in order to express the CRISPR endonuclease and/or a guide RNA complementary to an NRAS gene (in some embodiments, a variant NRAS gene found only in cancer cells). A recombinant nucleic acid construct may comprise a nucleic acid encoding a CRISPR endonuclease and/or a guide RNA complementary to an NRAS gene (in some embodiments, a variant NRAS gene found only in cancer cells), operably linked to a promoter suitable for expressing the CRISPR endonuclease and/or a guide RNA complementary to the NRAS gene (in some embodiments, the variant NRAS gene) in the cell. In some embodiments the nucleic acid encoding a CRISPR endonuclease is operably linked to the same promoter as the nucleic acid encoding the guide RNA. In other embodiments, the nucleic acid encoding a CRISPR endonuclease and the nucleic acid encoding the guide RNA are operably linked to different promoters. In some embodiments, the nucleic acid encoding a CRISPR endonuclease and/or the nucleic acid encoding a guide RNA are operably linked to a lung specific promoter.

In some embodiments, one or more CRISPR endonucleases and one or more guide RNAs may be provided in combination in the form of ribonucleoprotein particles (RNPs). An RNP complex can be introduced into a subject by means of, e.g., injection, electroporation, nanoparticles, vesicles, and/or with the assistance of cell-penetrating peptides.

DNA vectors containing nucleic acids such as those described herein also are also provided. A “DNA vector” is a replicon, such as a plasmid, phage, or cosmid, into which another DNA segment may be inserted so as to bring about the replication of the inserted segment. Generally, a DNA vector is capable of replication when associated with the proper control elements. Suitable vector backbones include, for example, those routinely used in the art such as plasmids, viruses, artificial chromosomes, BACs, YACs, or PACs. The term “DNA vector” includes cloning and expression vectors, as well as viral vectors and integrating vectors. An “expression vector” is a vector that includes a regulatory region. A wide variety of host/expression vector combinations may be used to express the nucleic acid sequences described herein. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The DNA vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The DNA vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region (e.g. a promoter) and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Direct injection of adenoviral vectors into lung tumors has been a routine procedure in clinical trials evaluating gene therapy of lung cancer. Dong et al., J. Int. Med. Res. 36, 1273-1287 (2008); Li et al., Cancer Gene Ther. 20, 251-259 (2013); Zhou et al., Cancer Gene Ther. 23, 1-6 (2016). Vectors can also comprise other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al., BioTechniques, 34:167-71 (2003). A large variety of such vectors are known in the art and are generally available.

Suitable nucleic acid delivery systems include recombinant viral vector, typically sequence from at least one of an Ad, AAV, helper-dependent adenovirus, retrovirus, or hemagglutinating virus of Japan-liposome (HVJ) complex. In such cases, the viral vector comprises a strong eukaryotic promoter operably linked to the polynucleotide e.g., a cytomegalovirus (CMV) promoter. The recombinant viral vector can include one or more of the polynucleotides therein, in some embodiments about one polynucleotide.

In embodiments in which the polynucleotide is to be administered with a viral vector, use of a viral titer (i.e., refers to the concentration of infectious viral particles in a sample) about 1×10⁷ PFU/ml, about 1×10⁸ PFU/ml, about 1×10⁹ PFU/ml, about 1×10¹⁰ PFU/ml, about 1×10¹¹ PFU/ml, or about 1×10¹² PFU/ml.

In embodiments in which the polynucleotide is to be administered with a non-viral vector, use of between from about 0.1 ng to about 100.0 mg will often be useful e.g., about 0.1 ng to about 95.0 mg, about 0.1 ng to about 90.0 mg, about 0.1 ng to about 85.0 mg, about 0.1 ng to about 80.0 mg, about 0.1 ng to about 75.0 mg, about 0.1 ng to about 70.0 mg, about 0.1 ng to about 65.0 mg, about 0.1 ng to about 60.0 mg, about 0.1 ng to about 55.0 mg, about 0.1 ng to about 50.0 mg, about 0.1 ng to about 45.0 mg, about 0.1 ng to about 40.0 mg, about 0.1 ng to about 35.0 mg, about 0.1 ng to about 30.0 mg, about 0.1 ng to about 25.0 mg, about 0.1 ng to about 20.0 mg, about 0.1 ng to about 19.0 mg, about 0.1 ng to about 18.0 mg, about 0.1 ng to about 17.0 mg, about 0.1 ng to about 16.0 mg, about 0.1 ng to about 15.0 mg, about 0.1 ng to about 14.0 mg, about 0.1 ng to about 13.0 mg, about 0.1 ng to about 12.0 mg, about 0.1 ng to about 11.0 mg, about 0.1 ng to about 10.0 mg, about 0.1 ng to about 9.9 mg, about 0.1 ng to about 9.8 mg, about 0.1 ng to about 9.7 mg, about 0.1 ng to about 9.6 mg, about 0.1 ng to about 9.5 mg, about 0.1 ng to about 9.4 mg, about 0.1 ng to about 9.3 mg, about 0.1 ng to about 9.2 mg, about 0.1 ng to about 9.1 mg, about 0.1 ng to about 9.0 mg, about 0.1 ng to about 8.9 mg, about 0.1 ng to about 8.8 mg, about 0.1 ng to about 8.7 mg, about 0.1 ng to about 8.6 mg, about 0.1 ng to about 8.5 mg, about 0.1 ng to about 8.4 mg, about 0.1 ng to about 8.3 mg, about 0.1 ng to about 8.2 mg, about 0.1 ng to about 8.1 mg, about 8.0 mg, about 0.1 ng to about 7.9 mg, about 0.1 ng to about 7.8 mg, about 0.1 ng to about 7.7 mg, about 0.1 ng to about 7.6 mg, about 0.1 ng to about 7.5 mg, about 0.1 ng to about 7.4 mg, about 0.1 ng to about 7.3 mg, about 0.1 ng to about 7.2 mg, about 0.1 ng to about 7.1 mg, about 7.0 mg, about 0.1 ng to about 6.9 mg, about 0.1 ng to about 6.8 mg, about 0.1 ng to about 6.7 mg, about 0.1 ng to about 6.6 mg, about 0.1 ng to about 6.5 mg, about 0.1 ng to about 6.4 mg, about 0.1 ng to about 6.3 mg, about 0.1 ng to about 6.2 mg, about 0.1 ng to about 6.1 mg, about 6.0 mg, about 0.1 ng to about 5.9 mg, about 0.1 ng to about 5.8 mg, about 0.1 ng to about 5.7 mg, about 0.1 ng to about 5.6 mg, about 0.1 ng to about 5.5 mg, about 0.1 ng to about 5.4 mg, about 0.1 ng to about 5.3 mg, about 0.1 ng to about 5.2 mg, about 0.1 ng to about 5.1 mg, about 5.0 mg, about 0.1 ng to about 4.9 mg, about 0.1 ng to about 4.8 mg, about 0.1 ng to about 4.7 mg, about 0.1 ng to about 4.6 mg, about 0.1 ng to about 4.5 mg, about 0.1 ng to about 4.4 mg, about 0.1 ng to about 4.3 mg, about 0.1 ng to about 4.2 mg, about 0.1 ng to about 4.1 mg, about 0.1 ng to about 4.0 mg, about 0.1 ng to about 3.9 mg, about 0.1 ng to about 3.8 mg, about 0.1 ng to about 3.7 mg, about 0.1 ng to about 3.6 mg, about 0.1 ng to about 3.5 mg, about 0.1 ng to about 3.4 mg, about 0.1 ng to about 3.3 mg, about 0.1 ng to about 3.2 mg, about 0.1 ng to about 3.1 mg, about 0.1 ng to about 2.0 mg, about 0.1 ng to about 2.9 mg, about 0.1 ng to about 2.8 mg, about 0.1 ng to about 2.7 mg, about 0.1 ng to about 2.6 mg, about 0.1 ng to about 2.5 mg, about 0.1 ng to about 2.4 mg, about 0.1 ng to about 2.3 mg, about 0.1 ng to about 2.2 mg, about 0.1 ng to about 2.1 mg, about 0.1 ng to about 2.0 mg, about 0.1 ng to about 1.9 mg, about 0.1 ng to about 1.8 mg, about 0.1 ng to about 1.7 mg, about 0.1 ng to about 1.6 mg, about 0.1 ng to about 1.5 mg, about 0.1 ng to about 1.4 mg, about 0.1 ng to about 1.3 mg, about 0.1 ng to about 1.2 mg, about 0.1 ng to about 1.1 mg, about 0.1 ng to about 1.0 mg, about 0.1 ng to about 900 μg, about 0.1 ng to about 800 μg, about 0.1 ng to about 700 μg, about 0.1 ng to about 600 μg, about 0.1 ng to about 500 μg, about 0.1 ng to about 400 μg, about 0.1 ng to about 300 μg, about 0.1 ng to about 200 μg, about 0.1 ng to about 100 μg, about 0.1 ng to about 90 μg, about 0.1 ng to about 80 μg, about 0.1 ng to about 70 μg, about 0.1 ng to about 60 μg, about 0.1 ng to about 50 μg, about 0.1 ng to about 40 μg, about 0.1 ng to about 30 μg, about 0.1 ng to about 20 μg, about 0.1 ng to about 10 μg, about 0.1 ng to about 1 μg, about 0.1 ng to about 900 ng, about 0.1 ng to about 800 ng, about 0.1 ng to about 700 ng, about 0.1 ng to about 600 ng, about 0.1 ng to about 500 ng, about 0.1 ng to about 400 ng, about 0.1 ng to about 300 ng, about 0.1 ng to about 200 ng, about 0.1 ng to about 100 ng, about 0.1 ng to about 90 ng, about 0.1 ng to about 80 ng, about 0.1 ng to about 70 ng, about 0.1 ng to about 60 ng, about 0.1 ng to about 50 ng, about 0.1 ng to about 40 ng, about 0.1 ng to about 30 ng, about 0.1 ng to about 20 ng, about 0.1 ng to about 10 ng, about 0.1 ng to about 1 ng, about 1 ng to about 4000 μg, about 1.0 ng to about 9.9 mg, about 0.1 ng to about 9.8 mg, about 1.0 ng to about 9.7 mg, about 1.0 ng to about 9.6 mg, about 1.0 ng to about 9.5 mg, about 1.0 ng to about 9.4 mg, about 1.0 ng to about 9.3 mg, about 1.0 ng to about 9.2 mg, about 1.0 ng to about 9.1 mg, about 1.0 ng to about 9.0 mg, about 1.0 ng to about 8.9 mg, about 1.0 ng to about 8.8 mg, about 1.0 ng to about 8.7 mg, about 1.0 ng to about 8.6 mg, about 1.0 ng to about 8.5 mg, about 1.0 ng to about 8.4 mg, about 1.0 ng to about 8.3 mg, about 1.0 ng to about 8.2 mg, about 1.0 ng to about 8.1 mg, about 8.0 mg, about 1.0 ng to about 7.9 mg, about 1.0 ng to about 7.8 mg, about 1.0 ng to about 7.7 mg, about 1.0 ng to about 7.6 mg, about 1.0 ng to about 7.5 mg, about 1.0 ng to about 7.4 mg, about 1.0 ng to about 7.3 mg, about 1.0 ng to about 7.2 mg, about 1.0 ng to about 7.1 mg, about 7.0 mg, about 1.0 ng to about 6.9 mg, about 1.0 ng to about 6.8 mg, about 1.0 ng to about 6.7 mg, about 1.0 ng to about 6.6 mg, about 1.0 ng to about 6.5 mg, about 1.0 ng to about 6.4 mg, about 1.0 ng to about 6.3 mg, about 1.0 ng to about 6.2 mg, about 1.0 ng to about 6.1 mg, about 6.0 mg, about 1.0 ng to about 5.9 mg, about 1.0 ng to about 5.8 mg, about 1.0 ng to about 5.7 mg, about 1.0 ng to about 5.6 mg, about 1.0 ng to about 5.5 mg, about 1.0 ng to about 5.4 mg, about 1.0 ng to about 5.3 mg, about 1.0 ng to about 5.2 mg, about 1.0 ng to about 5.1 mg, about 5.0 mg, about 1.0 ng to about 4.9 mg, about 1.0 ng to about 4.8 mg, about 1.0 ng to about 4.7 mg, about 1.0 ng to about 4.6 mg, about 1.0 ng to about 4.5 mg, about 1.0 ng to about 4.4 mg, about 1.0 ng to about 4.3 mg, about 1.0 ng to about 4.2 mg, about 1.0 ng to about 4.1 mg, about 1.0 ng to about 4.0 mg, about 1.0 ng to about 3.9 mg, about 1.0 ng to about 3.8 mg, about 1.0 ng to about 3.7 mg, about 1.0 ng to about 3.6 mg, about 1.0 ng to about 3.5 mg, about 1.0 ng to about 3.4 mg, about 1.0 ng to about 3.3 mg, about 1.0 ng to about 3.2 mg, about 1.0 ng to about 3.1 mg, about 1.0 ng to about 2.0 mg, about 1.0 ng to about 2.9 mg, about 1.0 ng to about 2.8 mg, about 1.0 ng to about 2.7 mg, about 1.0 ng to about 2.6 mg, about 1.0 ng to about 2.5 mg, about 1.0 ng to about 2.4 mg, about 1.0 ng to about 2.3 mg, about 1.0 ng to about 2.2 mg, about 1.0 ng to about 2.1 mg, about 1.0 ng to about 2.0 mg, about 1.0 ng to about 1.9 mg, about 1.0 ng to about 1.8 mg, about 1.0 ng to about 1.7 mg, about 1.0 ng to about 1.6 mg, about 1.0 ng to about 1.5 mg, about 1.0 ng to about 1.4 mg, about 1.0 ng to about 1.3 mg, about 1.0 ng to about 1.2 mg, about 1.0 ng to about 1.1 mg, about 1.0 ng to about 1.0 mg, about 1.0 ng to about 900 μg, about 1.0 ng to about 800 μg, about 1.0 ng to about 700 μg, about 1.0 ng to about 600 μg, about 1.0 ng to about 500 μg, about 1.0 ng to about 400 μg, about 1.0 ng to about 300 μg, about 1.0 ng to about 200 μg, about 1.0 ng to about 1.000 μg, about 1.0 ng to about 90 μg, about 1.0 ng to about 80 μg, about 1.0 ng to about 70 μg, about 1.0 ng to about 60 μg, about 1.0 ng to about 50 μg, about 1.0 ng to about 40 μg, about 1.0 ng to about 30 μg, about 1.0 ng to about 20 μg, about 1.0 ng to about 10 μg, about 1.0 ng to about 1.0 μg, about 1.0 ng to about 900 ng, about 1.0 ng to about 800 ng, about 1.0 ng to about 700 ng, about 1.0 ng to about 600 ng, about 1.0 ng to about 500 ng, about 1.0 ng to about 400 ng, about 1.0 ng to about 300 ng, about 1.0 ng to about 200 ng, about 1.0 ng to about 100 ng, about 1.0 ng to about 90 ng, about 1.0 ng to about 80 ng, about 1.0 ng to about 70 ng, about 1.0 ng to about 60 ng, about 1.0 ng to about 50 ng, about 1.0 ng to about 40 ng, about 1.0 ng to about 30 ng, about 1.0 ng to about 20 ng, about 1.0 ng to about 10 ng, about 10 ng to about 100.0 mg, about 20 ng to about 100.0 mg, about 30 ng to about 100.0 mg, about 40 ng to about 100.0 mg, about 50 ng to about 100.0 mg, about 60 ng to about 100.0 mg, about 70 ng to about 100.0 mg, about 80 ng to about 100.0 mg, about 90 ng to about 100.0 mg, about 100 ng to about 100.0 mg, about 200 ng to about 100.0 mg, about 300 ng to about 100.0 mg, about 400 ng to about 100.0 mg, about 500 ng to about 100.0 mg, about 600 ng to about 100.0 mg, about 700 ng to about 100.0 mg, about 800 ng to about 100.0 mg, about 900 ng to about 100.0 mg, about 1 μg to about 100.0 mg, 10 μg to about 100.0 mg, 20 μg to about 100.0 mg, 30 μg to about 100.0 mg, 40 μg to about 100.0 mg g, 50 μg to about 100.0 mg, 60 μg to about 100.0 mg, 70 μg to about 100.0 mg, 80 μg to about 100.0 mg, 90 μg to about 100.0 mg, 100 μg to about 100.0 mg, 200 μg to about 100.0 mg, 300 μg to about 100.0 mg, 400 μg to about 100.0 mg, 500 μg to about 100.0 mg, 600 μg to about 100.0 mg, 700 μg to about 100.0 mg, 800 μg to about 100.0 mg, 900 μg to about 100.0 mg, 1.0 mg to about 100.0 mg, 1.1 mg to about 100.0 mg, 1.2 mg to about 100.0 mg, 1.3 mg to about 100.0 mg, 1.4 mg to about 100.0 mg, 1.5 mg to about 100.0 mg, 1.6 mg to about 100.0 mg, 1.7 mg to about 100.0 mg, 1.8 mg to about 100.0 mg, 1.9 mg to about 100.0 mg, 2.0 mg to about 100.0 mg, 2.1 mg to about 100.0 mg, 2.2 mg to about 100.0 mg, 2.3 mg to about 100.0 mg, 2.4 mg to about 100.0 mg, 2.5 mg to about 100.0 mg, 2.6 mg to about 100.0 mg, 2.7 mg to about 100.0 mg, 2.8 mg to about 100.0 mg, 2.9 mg to about 100.0 mg, 3.0 mg to about 100.0 mg, 3.1 mg to about 100.0 mg, 3.2 mg to about 100.0 mg, 3.3 mg to about 100.0 mg, 3.4 mg to about 100.0 mg, 3.5 mg to about 100.0 mg, 3.6 mg to about 100.0 mg, 3.7 mg to about 100.0 mg, 3.8 mg to about 100.0 mg, 3.9 mg to about 100.0 mg, 4.0 mg to about 100.0 mg, 4.1 mg to about 100.0 mg, 4.2 mg to about 100.0 mg, 4.3 mg to about 100.0 mg, 4.4 mg to about 100.0 mg, 4.5 mg to about 100.0 mg, 4.6 mg to about 100.0 mg, 4.7 mg to about 100.0 mg, 4.8 mg to about 100.0 mg, 4.9 mg to about 100.0 mg, 5.0 mg to about 100.0 mg, 5.1 mg to about 100.0 mg, 5.2 mg to about 100.0 mg, 5.3 mg to about 100.0 mg, 5.4 mg to about 100.0 mg, 5.5 mg to about 100.0 mg, 5.6 mg to about 100.0 mg, 5.7 mg to about 100.0 mg, 5.8 mg to about 100.0 mg, 5.9 mg to about 100.0 mg, 6.0 mg to about 100.0 mg, 6.1 mg to about 100.0 mg, 6.2 mg to about 100.0 mg, 6.3 mg to about 100.0 mg, 6.4 mg to about 100.0 mg, 6.5 mg to about 100.0 mg, 3.6 mg to about 100.0 mg, 6.7 mg to about 100.0 mg, 6.8 mg to about 100.0 mg, 6.9 mg to about 100.0 mg, 7.0 mg to about 100.0 mg, 7.1 mg to about 100.0 mg, 7.2 mg to about 100.0 mg, 7.3 mg to about 100.0 mg, 7.4 mg to about 100.0 mg, 7.5 mg to about 100.0 mg, 7.6 mg to about 100.0 mg, 7.7 mg to about 100.0 mg, 7.8 mg to about 100.0 mg, 7.9 mg to about 100.0 mg, 8.0 mg to about 100.0 mg, 8.1 mg to about 100.0 mg, 8.2 mg to about 100.0 mg, 8.3 mg to about 100.0 mg, 8.4 mg to about 100.0 mg, 8.5 mg to about 100.0 mg, 8.6 mg to about 100.0 mg, 8.7 mg to about 100.0 mg, 8.8 mg to about 100.0 mg, 8.9 mg to about 100.0 mg, 9.0 mg to about 100.0 mg, 9.1 mg to about 100.0 mg, 9.2 mg to about 100.0 mg, 9.3 mg to about 100.0 mg, 9.4 mg to about 100.0 mg, 9.5 mg to about 100.0 mg, 9.6 mg to about 100.0 mg, 9.7 mg to about 100.0 mg, 9.8 mg to about 100.0 mg, 9.9 mg to about 100.0 mg, about 10.0 mg to about 100.0 mg, about 11.0 mg to about 100.0 mg, about 12.0 mg to about 100.0 mg, about 13.0 mg to about 100.0 mg, about 14.0 mg to about 100.0 mg, about 15.0 mg to about 100.0 mg, about 16.0 mg to about 100.0 mg, about 17.0 mg to about 100.0 mg, about 18.0 mg to about 100.0 mg, about 19.0 mg to about 100.0 mg, about 20.0 mg to about 100.0 mg, about 25.0 mg to about 100.0 mg, about 30.0 mg to about 100.0 mg, about 35.0 mg to about 100.0 mg, about 40.0 mg to about 100.0 mg, about 45.0 mg to about 100.0 mg, about 50.0 mg to about 100.0 mg, about 55.0 mg to about 100.0 mg, about 60.0 mg to about 100.0 mg, about 65.0 mg to about 100.0 mg, about 70.0 mg to about 100.0 mg, about 75.0 mg to about 100.0 mg, about 80.0 mg to about 100.0 mg, about 85.0 mg to about 100.0 mg, about 90.0 mg to about 100.0 mg, or about 95.0 mg to about 100.0 mg.

Additional vectors include viral vectors, fusion proteins and chemical conjugates. Retroviral vectors include Moloney murine leukemia viruses and HIV-based viruses. One HIV-based viral vector comprises at least two vectors wherein the gag and pol genes are from an HIV genome and the env gene is from another virus. DNA viral vectors include pox vectors such as orthopox or avipox vectors, herpesvirus vectors such as a herpes simplex I virus (HSV) vector (Geller et al., J. Neurochem 64:487 (1995); Lim et al., in DNA Cloning: Mammalian Systems, D. Glover, Ed. (Oxford Univ. Press, Oxford England) (1995); Geller et al., Proc Natl. Acad. Sci. U.S.A. 90:7603 (1993); Geller et al., Proc Natl. Acad. Sci USA 87:1149 (1990)), Ad Vectors (LaSalle et al., Science 259:988 (1993); Davidson et al., Nat. Genet. 3:219 (1993); Yang et al., J. Virol. 69:2004 (1995)), and AAV Vectors (Kaplitt et al., Nat. Genet. 8:148 (1994)).

If desired, the polynucleotides described here may also be used with a microdelivery vehicle such as cationic liposomes, adenoviral vectors, and exosomes. For a review of the procedures for liposome preparation, targeting and delivery of contents, see Mannino and Gould-Fogerite, BioTechniques 6:682 (1988). See also, Feigner and Holm, Bethesda Res. Lab. Focus 11:21 (1989) and Maurer, Bethesda Res. Lab. Focus 11:25 (1989). In some embodiments, exosomes may be used for delivery of a nucleic acid encoding a CRISPR endonuclease and/or guide RNA to a target cell, e.g. a cancer cell. Exosomes are nanosized vesicles secreted by a variety of cells and are comprised of cellular membranes. Exosomes can attach to target cells by a range of surface adhesion proteins and vector ligands (tetraspanins, integrins, CD11b and CD18 receptors), and deliver their payload to target cells. Several studies indicate that exosomes have a specific cell tropism, according to their characteristics and origin, which can be used to target them to disease tissues and/or organs. See Batrakova et al., J. Control. Release 219:396-405 (2015). For example, cancer-derived exosomes function as natural carriers that can efficiently deliver CRISPR/Cas9 plasmids to cancer cells. See Kim et al., J. Control. Release 266:8-16 (2017).

Replication-defective recombinant adenoviral vectors, can be produced in accordance with known techniques. See Quantin et al., Proc. Natl. Acad. Sci. USA 89:2581-84 (1992); Stratford-Perricadet et al., J. Clin. Invest. 90:626-30 (1992); and Rosenfeld et al., Cell 68143-55 (1992).

Another delivery method is to use single stranded DNA producing vectors which can produce the expressed products intracellularly. See, e.g., Chen et al., BioTechniques 34:167-71 (2003).

Introduction of CRISPR/Cas systems can be accomplished by lipid nanoparticle (LNP)-mediated delivery. For example, LNP-mediated delivery can be used to deliver a combination of Cas mRNA and guide RNA or a combination of Cas protein and guide RNA. Delivery through such methods results in transient Cas expression, and the biodegradable lipids improve clearance, improve tolerability, and decrease immunogenicity. Lipid formulations can protect biological molecules from degradation while improving their cellular uptake.

LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840, herein incorporated by reference in its entirety for all purposes. An exemplary lipid nanoparticle can comprise a cationic lipid and one or more other components. In one embodiment, the other component can comprise a helper lipid such as cholesterol. In another embodiment, the other components can comprise a helper lipid such as cholesterol and a neutral lipid such as distearoylphosphatidylcholine (DSPC).

An LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al., Cell Rep. 22:1-9 (2018) and WO 2017/173054, each of which is herein incorporated by reference in its entirety for all purposes. In certain LNPs, the cargo can include a guide RNA or a nucleic acid encoding a guide RNA. In certain LNPs, the cargo can include an exogenous donor nucleic acid. In certain LNPs, the cargo can include a guide RNA or a nucleic acid encoding a guide RNA and a Cas protein or a nucleic acid encoding a Cas protein. In certain LNPs, the cargo can include a guide RNA or a nucleic acid encoding a guide RNA, a Cas protein or a nucleic acid encoding a Cas protein, and an exogenous donor nucleic acid.

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy-)carbonyl)oxy)methyl)propyl octadeca-9,12-dienoate, also called 3-((4,4-bis(octyloxy)butanoyl)oxy)-2-((((3-(diethylamino)propoxy)carbonyl-)oxy)methyl)propyl (9Z,12Z)-octadeca-9,12-dienoate. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1-,3-diyl(9Z,9Z′,12Z,12Z′)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as Dlin-MC3-DMA (MC3))).

Cationic lipid can be present in embodiments of the composition and lipid particles can comprise an amount from about 30 to about 60 mole percent (“mol %”, or the percentage of the total moles that is of a particular component), from about 30 mol % to about 55 mol %, from about 30 mol % to about 50 mol %, from about 30 mol % to about 45 mol %, from about 30 mol % to about 40 mol %, from about 30 mol % to about 35 mol %, from about 35 mol % to about 60 mol %, from about 40 mol % to about 60 mol %, from about 45 mol % to about 60 mol %, from about 50 mol % to about 60 mol %, from about 55 mol % to about 60 mol %, from about 35 mol % to about 55 mol %, from about 40 mol % to about 50 mol %. In in some embodiments, the cationic lipid is present in about 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral (also termed structural) lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, and combinations thereof. For example, the neutral phospholipid may be selected from the group consisting of distearoylphosphatidylcholine (DSPC) and dimyristoyl phosphatidyl ethanolamine (DMPE).

In certain embodiments, a neutral lipid is present in the lipid particle in an amount from about 20 mol % to about 40 mol %, from about 20 mol % to about 35 mol %, from about 20 mol % to about 30 mol %, from about 20 mol % to about 25 mol %, from about 25 mol % to about 40 mol %, from about 30 mol % to about 40 mol %, from about 30 mol % to about 40 mol %, from about 35 mol % to about 40 mol %, from about 25 mol % to about 35 mol %. In in some embodiments, the cationic lipid is present in about 20, 21, 22, 23, 24, 25, 26, 27, 28, 29, 30, 31, 32, 33, 34, 35, 36, 37, 38, 39, or 40 mol %.

In some embodiments, the lipids can be any of the lipids disclosed in US20210251898, US20210220449, US20210128488, US20210122703, US20210122702, US20210113483, US20210107861, US20210095309, US20210087135, US20190292566 each incorporated herein by reference in its entirety.

Commercially available LNPs include, e.g., Lipofectamine™ CRISPRMAX™ Cas9 Transfection Reagent (available from ThermoFisher Scientific, Waltham, MA), Pro-DeliverlN™ CRISPR Transfection Reagent (available from Oz Biosciences, San Diego, CA), and NanoAssemblr® LNPs (available from Precision NanoSystems, Vancouver, BC).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety. The hydrophilic head group of stealth lipid can comprise, for example, a polymer moiety selected from polymers based on poly(ethylene glycol), poly(oxazoline), poly(vinyl alcohol), poly(glycerol), poly(N-vinylpyrrolidone), polyaminoacids, and poly N-(2-hydroxypropyl)methacrylamide.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

In some embodiments, the stealth lipid may be PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-ω-methyl-poly(ethylene glycol), PEG-DMB (3,4-ditetradecoxylbenzyl-ω-methyl-poly(ethylene glycol)ether), 1,2-dimyristoyl-sn-glycero-3-phosphoethanolamine-N-[methoxy(polyethylene glycol)-2000](PEG2k-DMG), 1,2-distearoyl-sn-glycero-3-phosphoethanolamine-N-[methoxy(polyethylene glycol)-2000](PEG2k-DSPE), 1,2-distearoyl-sn-glycerol, methoxypoly ethylene glycol (PEG2k-DSG), poly(ethylene glycol)-2000-dimethacrylate (PEG2k-DMA), or 1,2-distearyloxypropyl-3-amine-N-[methoxy(polyethylene glycol)-2000] (PEG2k-DSA).

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5.

In some LNPs, the cargo can comprise Cas mRNA and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25.

In some LNPs, the cargo can comprise exogenous donor nucleic acid and gRNA. The exogenous donor nucleic acid and gRNAs can be in different ratios. For example, the LNP formulation can include a ratio of exogenous donor nucleic acid to gRNA nucleic acid ranging from about 25:1 to about 1:25, ranging from about 10:1 to about 1:10, ranging from about 5:1 to about 1:5, or about 1:1. Alternatively, the LNP formulation can include a ratio of exogenous donor nucleic acid to gRNA nucleic acid from about 1:1 to about 1:5, about 5:1 to about 1:1, about 10:1, or about 1:10. Alternatively, the LNP formulation can include a ratio of exogenous donor nucleic acid to gRNA nucleic acid of about 1:10, 25:1, 10:1, 5:1, 3:1, 2.5:1, 1:1, 1:2.5, 1:3, 1:5, 1:10, or 1:25.

In some embodiments, one or more CRISPR endonucleases and one or more guide RNAs may be provided in combination in the form of ribonucleoprotein particles (RNPs). An RNP complex can be introduced into a subject by means of, e.g., injection, electroporation, nanoparticles (including, e.g., lipid nanoparticles), vesicles, and/or with the assistance of cell-penetrating peptides. See, e.g., Lin et al., ELife 3:e04766 (2014); Sansbury et al., CRISPR J. 2:121-32 (2019); US2019/0359973).

LNP particles can have a diameter of less than 1000 nm, 500 nm, 250 nm, 200 nm, 150 nm, 100 nm, 75 nm, 50 nm, or 25 nm. Alternatively, a nanoparticle may range in size from 1-1000 nm, 1-500 nm, 1-250 nm, 25-200 nm, 25-100 nm, 35-75 nm, or 25-60 nm. LNPs may be made from cationic, anionic, or neutral lipids. Neutral lipids, such as the fusogenic phospholipid DOPE or the membrane component cholesterol, may be included in LNPs as “helper lipids” to enhance transfection activity and nanoparticle stability. LNPs may also be comprised of hydrophobic lipids, hydrophilic lipids, or both hydrophobic and hydrophilic lipids.

In certain embodiments, the cationic lipid N-[1-(2,3-dioleyloxy)propyl]-N,N,N-trimethylammonium chloride (DOTMA) can be used. DOTMA can be formulated alone or combined with the neutral lipid, dioleoylphosphatidyl-ethanolamine (DOPE) or other cationic or non-cationic lipids into a liposomal transfer vehicle or a lipid nanoparticle, and such liposomes can be used to enhance the delivery of nucleic acids into target cells. Other suitable cationic lipids include, but are not limited to, 5-carboxyspermylglycinedioctadecylamide, 2,3-dioleyloxy-N-[2(spermine-carboxamido)ethyl]-N,N-dimethyl-1-propanaminium, 1,2-Dioleoyl-3-Dimethylammonium-Propane, 1,2-Dioleoyl-3-Trimethylammonium-Propane. Contemplated cationic lipids also include 1,2-distearyloxy-N,N-dimethyl-3-aminopropane, 1,2-dioleyloxy-N,N-dimethyl-3-aminopropane, 1,2-dilinoleyloxy-N,N-dimethyl-3-aminopropane, 1,2-dilinolenyloxy-N,N-dimethyl-3-aminopropane, N-dioleyl-N,N-dimethylammonium chloride, N,N-distearyl-N,N-dimethylammonium bromide, N-(1,2-dimyristyloxyprop-3-yl)-N,N-dimethyl-N-hydroxyethyl ammonium bromide, 3-dimethylamino-2-(cholest-5-en-3-beta-oxybutan-4-oxy)-1-(cis,cis-9,12-octadecadienoxy)propane, 2-[5′-(cholest-5-en-3-beta-oxy)-3′-oxapentoxy)-3-dimethyl-1-(cis,cis-9′,12′-octadecadienoxy)propane, N,N-dimethyl-3,4-dioleyloxybenzylamine, 1,2-N,N′-dioleylcarbamyl-3-dimethylaminopropane, 2,3-Dilinoleoyloxy-N,N-dimethylpropylamine, 1,2-N,N′-Dilinoleylcarbamyl-3-dimethylaminopropane, 1,2-Dilinoleoylcarbamyl-3-dimethylaminopropane, 2,2-dilinoleyl-4-dimethylaminomethyl-[1,3]-dioxolane, 2,2-dilinoleyl-4-dimethylaminoethyl-[1,3]-dioxolane, and 2-(2,2-di((9Z,12Z)-octadeca-9,12-dien-1-yl)-1,3-dioxolan-4-yl)-N,N-dimethylethanamine (DLin-KC2-DMA)), or mixtures thereof.

In some embodiments, non-cationic lipids can be used. As used herein, the phrase “non-cationic lipid” refers to any neutral, zwitterionic, or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), DOPE, palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone or can be used in combination with other excipients, for example, cationic lipids.

The term “chemoresistance” is meant a cancer cell, a cancer, or a cancerous tumor that fails to respond to treatment by conventional anti-cancer agents. Chemoresistance may be characterized by recurrence or continuation of cancer growth following a treatment regimen with anti-cancer agents. Chemoresistant cancer cells, cancers, or cancerous tumors may be chemoresistant as a result of, for example, an alteration in cellular transcriptional programs that control cell metabolism, resistance to stress, epithelial-to-mesenchymal transition, cell cycle regulation, among others.

The term “anti-cancer agents” includes, but is not limited to, alkylating agents, anthracyclines, taxanes, epothilones, histone deacetylase inhibitors, inhibitors of topoisomerase I or II, kinase inhibitors, nucleotide analogs, precursor analogs, peptides, antibodies, platinum-based agents, retinoids, vinca alkaloids, and derivatives thereof.

### NRAS

In some embodiments, a human NRAS gene refers to the gene described by NCBI Entrez Gene ID No. 4893, including mutants and variants thereof. In other embodiments, the NRAS gene is from one of the following organisms: mouse (see, e.g., NCBI Entrez Gene ID No. 18176), rat (see, e.g., NCBI Entrez Gene ID No. 24605), fish (see, e.g., NCBI Entrez Gene ID No. 30380), dog (see, e.g., NCBI Entrez Gene ID No. 403872), cattle (see, e.g., NCBI Entrez Gene ID No. 506322), horse (see, e.g., NCBI Entrez Gene ID No. 100059469), or chimpanzee (see, e.g., NCBI Entrez Gene ID No. 742713). In some embodiments, the human NRAS gene has the sequence set forth in RefSeq NM_002524 (SEQ ID NO:5 below), which encodes the human nras protein having the amino acid sequence set forth in RefSeq NP_002515 (SEQ ID NO:6 below). The coding region of RefSeq NM_002524 is found at bases 138-567.

SEQ ID NO: 5
ggggccggaa gtgccgctcc ttggtggggg ctgttcatgg cggttccggg gtctccaaca tttttcccgg
ctgtggtcct aaatctgtcc aaagcagagg cagtggagct tgaggttctt gctggtgtga aatgactgag
tacaaactgg tggtggttgg agcaggtggt gttgggaaaa gcgcactgac aatccagcta atccagaacc
actttgtaga tgaatatgat cccaccatag aggattctta cagaaaacaa gtggttatag atggtgaaac
ctgtttgttg gacatactgg atacagctgg acaagaagag tacagtgcca tgagagacca atacatgagg
acaggcgaag gcttcctctg tgtatttgcc atcaataata gcaagtcatt tgcggatatt aacctctaca
gggagcagat taagcgagta aaagactcgg atgatgtacc tatggtgcta gtgggaaaca agtgtgattt
gccaacaagg acagttgata caaaacaagc ccacgaactg gccaagagtt acgggattcc attcattgaa
acctcagcca agaccagaca gggtgttgaa gatgcttttt acacactggt aagagaaata cgccagtacc
gaatgaaaaa actcaacagc agtgatgatg ggactcaggg ttgtatggga ttgccatgtg tggtgatgta
acaagatact tttaaagttt tgtcagaaaa gagccacttt caagctgcac tgacaccctg gtcctgactt
ccctggagga gaagtattcc tgttgctgtc ttcagtctca cagagaagct cctgctactt ccccagctct cagtagttta
gtacaataat ctctatttga gaagttctca gaataactac ctcctcactt ggctgtctga ccagagaatg cacctcttgt
tactccctgt tatttttctg ccctgggttc ttccacagca caaacacacc tctgccaccc caggtttttc atctgaaaag
cagttcatgt ctgaaacaga gaaccaaacc gcaaacgtga aattctattg aaaacagtgt cttgagctct
aaagtagcaa ctgctggtga tttttttttt ctttttactg ttgaacttag aactatgcta atttttggag aaatgtcata
aattactgtt ttgccaagaa tatagttatt attgctgttt ggtttgttta taatgttatc ggctctattc tctaaactgg
catctgctct agattcataa atacaaaaat gaatactgaa ttttgagtct atcctagtct tcacaacttt gacgtaatta
aatccaactt tcacagtgaa gtgccttttt cctagaagtg gtttgtagac ttcctttata atatttcagt ggaatagatg
tctcaaaaat ccttatgcat gaaatgaatg tctgagatac gtctgtgact tatctaccat tgaaggaaag ctatatctat
ttgagagcag atgccatttt gtacatgtat gaaattggtt ttccagaggc ctgttttggg gctttcccag gagaaagatg
aaactgaaag cacatgaata atttcactta ataattttta cctaatctcc acttttttca taggttacta cctatacaat
gtatgtaatt tgtttcccct agcttactga taaacctaat attcaatgaa cttccatttg tattcaaatt tgtgtcatac
cagaaagctc tacatttgca gatgttcaaa tattgtaaaa ctttggtgca ttgttattta atagctgtga tcagtgattt
tcaaacctca aatatagtat attaacaaat tacattttca ctgtatatca tggtatctta atgatgtata taattgcctt
caatcccctt ctcaccccac cctctacagc ttcccccaca gcaatagggg cttgattatt tcagttgagt
aaagcatggt gctaatggac cagggtcaca gtttcaaaac ttgaacaatc cagttagcat cacagagaaa
gaaattcttc tgcatttgct cattgcacca gtaactccag ctagtaattt tgctaggtag ctgcagttag ccctgcaagg
aaagaagagg tcagttagca caaacccttt accatgactg gaaaactcag tatcacgtat ttaaacattt ttttttcttt
tagccatgta gaaactctaa attaagccaa tattctcatt tgagaatgag gatgtctcag ctgagaaacg
ttttaaattc tctttattca taatgttctt tgaagggttt aaaacaagat gttgataaat ctaagctgat gagtttgctc
aaaacaggaa gttgaaattg ttgagacagg aatggaaaat ataattaatt gatacctatg aggatttgga
ggcttggcat tttaatttgc agataatacc ctggtaattc tcatgaaaaa tagacttgga taacttttga taaaagacta
attccaaaat ggccactttg ttcctgtctt taatatctaa atacttactg aggtcctcca tcttctatat tatgaatttt
catttattaa gcaaatgtca tattaccttg aaattcagaa gagaagaaac atatactgtg tccagagtat
aatgaacctg cagagttgtg cttcttactg ctaattctgg gagctttcac agtactgtca tcatttgtaa atggaaattc
tgcttttctg tttctgctcc ttctggagca gtgctactct gtaattttcc tgaggcttat cacctcagtc atttcttttt
taaatgtctg tgactggcag tgattctttt tcttaaaaat ctattaaatt tgatgtcaaa ttagggagaa agatagttac
tcatcttggg ctcttgtgcc aatagccctt gtatgtatgt acttagagtt ttccaagtat gttctaagca cagaagtttc
taaatggggc caaaattcag acttgagtat gttctttgaa taccttaaga agttacaatt agccgggcat
ggtggcccgt gcctgtagtc ccagctactt gagaggctga ggcaggagaa tcacttcaac ccaggaggtg
gaggttacag tgagcagaga tcgtgccact gcactccagc ctgggtgaca agagagactt gtctccaaaa
aaaaagttac acctaggtgt gaattttggc acaaaggagt gacaaactta tagttaaaag ctgaataact
tcagtgtggt ataaaacgtg gtttttaggc tatgtttgtg attgctgaaa agaattctag tttacctcaa aatccttctc
tttccccaaa ttaagtgcct ggccagctgt cataaattac atattccttt tggttttttt aaaggttaca tgttcaagag
tgaaaataag atgttctgtc tgaaggctac catgccggat ctgtaaatga acctgttaaa tgctgtattt
gctccaacgg cttactatag aatgttactt aatacaatat catacttatt acaattttta ctataggagt gtaataggta
aaattaatct ctattttagt gggcccatgt ttagtctttc accatccttt aaactgctgt gaattttttt gtcatgactt
gaaagcaagg atagagaaac actttagaga tatgtggggt ttttttacca ttccagagct tgtgagcata
atcatatttg ctttatattt atagtcatga actcctaagt tggcagctac aaccaagaac caaaaaatgg tgcgttctgc
ttcttgtaat tcatctctgc taataaatta taagaagcaa ggaaaattag ggaaaatatt ttatttggat ggtttctata
aacaagggac tataattctt gtacattatt tttcatcttt gctgtttctt tgagcagtct aatgtgccac acaattatct
aaggtatttg ttttctataa gaattgtttt aaaagtattc ttgttaccag agtagttgta ttatatttca aaacgtaaga
tgatttttaa aagcctgagt actgacctaa gatggaattg tatgaactct gctctggagg gaggggagga
tgtccgtgga agttgtaaga cttttatttt tttgtgccat caaatatagg taaaaataat tgtgcaattc tgctgtttaa
acaggaacta ttggcctcct tggccctaaa tggaagggcc gatattttaa gttgattatt ttattgtaaa ttaatccaac
ctagttcttt ttaatttggt tgaatgtttt ttcttgttaa atgatgttta aaaaataaaa actggaagtt cttggcttag
tcataa
SEQ ID NO: 6
mteyklvvvg aggvgksalt iqliqnhfvd eydptiedsy rkqvvidget clldildtag qeeysamrdq
ymrtgegflc vfainnsksf adinlyreqi krvkdsddvp mvlvgnkcdl ptrtvdtkqa helaksygip fietsaktrq
gvedafytlv reirqyrmkk lnssddgtqg cmglpcvvm

A variety of NRAS mutations associated with cancer are known and may be suitably detected by the methods described herein. The most frequent NRAS alterations observed in cancer are mutations at codons 12, 13, and 61 (90%), and within the phosphate binding loop/GI motif (residues 10-17), the switch II region (residues 59-67), and the G5 motif (residues 145-147). Somatic mutations in NRAS is rarely (0.2-1%) reported in primary NSCLC, but their role in carcinogenesis has been proven. Smoking and environmental carcinogens are associated with the etiology of NRAS mutated lung cancer. NRAS mutations have been correlated with metastases of NSCLC (1.5%). Somatic mutations in NRAS are generally associated with poor response to standard therapies. MEK inhibitors, such as selumetinib, are effective in treating cancer patients with RAS mutations. NRAS mutations, such as E63K, are associated with resistance to anti-EGFR therapies, such as cetuximab and panitumumab; anti-BRAF therapies, such as vemurafenib, dabrafenib, encorafenib, sorafenib, tivatinib, ARQ736, ARQ680, AZ628, CEP-32496, GDC-0879, NMS-P186, NMS-P349, NMS-P383, NMS-P396, NMS-P730, PLX3603, PLX4720, PF-04880594, PLX4734, RAF265, R04987655, SB590885, BMS908662, WYE-130600, TAK632, MLN 2480, XL281, LUT001, LUT156, LUT192, LUT195, or LUT197 (and, in some embodiments, one or more of any of these anti-BRAF therapies in combination with one or more of trametinib (a MEK inhibitor), cobimetinib (a MEK inhibitor), binimetinib (a MEK inhibitor), or cetuximab (an anti-EGFR antibody)); and radiotherapy.

The concentration of anti-cancer therapies can vary widely, and will be selected primarily based on fluid volumes, viscosities, body weight and the like in accordance with the particular mode of administration selected and the biological system's needs. Generally, the amount of the anti-cancer therapy or therapies present in a pharmaceutical composition will be that which will produce a therapeutic effect. For example, in some embodiments, the weight per volume (w/v) or weight percent (wt %) concentration of an anti-cancer therapy or therapies in a pharmaceutical composition may be between about 0.001% to 100%, 0.001% to 90%, 0.001% to 80%, 0.001% to 70%, 0.001% to 60%, 0.001% to 50%, 0.001% to 40%, 0.001% to 30%, 0.001% to 20%, 0.001% to 10%, 0.001% to 1%, 0.01% to 100%, 0.01% to 90%, 0.01% to 80%, 0.01% to 70%, 0.01% to 60%, 0.01% to 50%, 0.01% to 40%, 0.01% to 30%, 0.01% to 20%, 0.01% to 10%, 0.01% to 1%, 0.1% to 100%, 0.1% to 90%, 0.1% to 80%, 0.1% to 70%, 0.1% to 60%, 0.1% to 50%, 0.1% to 40%, 0.1% to 30%, 0.1% to 20%, 0.1% to 10%, 0.1% to 1%, 1% to 100%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, 0.1% to 0.9%, 0.1% to 0.8%, 0.1% to 0.7%, 0.1% to 0.6%, 0.1% to 0.5%, 0.1% to 0.4%, 0.1% to 0.3%, 0.1% to 0.2%, 0.2% to 1%, 0.3% to 1%, 0.4% to 1%, 0.5% to 1%, 0.6% to 1%, 0.7% to 1%, 0.8% to 1%, or 0.9% to 1%.

In other embodiments, the concentration of an anti-cancer therapy or therapies in a pharmaceutical composition may be about 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, or 1 M. In some aspects, the concentration (molarity or wt %) of an anti-cancer therapy or therapies that produces a therapeutic effect in a subject (e.g., a human or other mammal) can be extrapolated from in vitro or in vivo data, from cell culture and/or animal experiments.

In some embodiments, an NRAS mutation is selected from the group consisting of G12C, G12D, G12V, G12A, G12S, G12R, G13D, G13C, G13S, G13R, G13A, G13V, Q61H, Q61L, Q61R, Q61K, Q61P, Q61E, and a combination thereof.

By using a CRISPR/Cas system as described herein, in some embodiments, it is possible to target and knock out the mutated NRAS protein, while not disrupting the function of wildtype NRAS protein. Thus, some embodiments are directed to reducing or, in some embodiments, eliminating expression of variant NRAS found only on allele carrying an NRAS mutation. For example, the Q61K NRAS mutation results from a C>A mutation at position 181 of the NRAS coding sequence (position 318 of SEQ ID NO:5). This C>A mutation creates a PAM site for Cas12a in the Q61K NRAS that is not present in the wild-type NRAS sequence.

In some embodiments, a guide RNA is complementary to a variant NRAS gene that is found only in cancer cells and not in wild-type NRAS genes in normal (i.e., non-cancerous) cells. In some embodiments, introducing the one or more nucleic acid sequence(s) encoding the gRNA and the nucleic acid sequence encoding a CRISPR-associated endonuclease into the cell reduces variant NRAS expression and/or activity in the cell, but does not completely eliminate it. In other embodiments, variant NRAS expression and/or activity in the cell are completely eliminated.

In some embodiments, an NRAS gene or protein shares a percent sequence identity with the SEQ ID NO:5 and 6, respectively. “Percent sequence identity” refers to the degree of sequence identity between any given reference sequence, e.g., SEQ ID NO: 5 or 6, and a variant NRAS gene or nras protein. A candidate sequence typically has a length that is from 80% to 200% of the length of the reference sequence, e.g., 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 percent of the length of the reference sequence. A percent identity for any candidate nucleic acid or polypeptide relative to a reference nucleic acid or polypeptide can be determined as follows. A reference sequence (e.g., a nucleic acid sequence or an amino acid sequence) can be aligned to one or more candidate sequences using, e.g., ClustalW, Clustal Ω, or BLAST, which allows alignments of nucleic acid or polypeptide sequences to be carried out across their entire length (Chenna et al., Nucleic Acids Res. 31:3497-3500 (2003)).

Typically, when an alignment is prepared based upon an amino acid sequence, the alignment contains insertions and deletions which are so identified with respect to a reference sequence and the numbering of the amino acid residues is based upon a reference scale provided for the alignment. However, any given reference sequence may have fewer amino acid residues than the reference scale. Herein, when discussing the parental sequence, the term “the same position” or the “corresponding position” refers to the amino acid located at the same residue number in each of the sequences, with respect to the reference scale for the aligned sequences. However, when taken out of the alignment, each of the proteins may have these amino acids located at different residue numbers.

Variants of a polypeptide (e.g., of nras) may also refer to a polypeptide comprising a referenced amino acid sequence except for one or more (e.g., 1, 2, 3, 4, 5, 6, 7, 8, 9 or 10) mutations such as, for example, missense mutations (e.g., conservative substitutions), nonsense mutations, deletions, or insertions.

### BRAF

BRAF is a member of the Raf kinase family of serine/threonine-specific protein kinases. This protein plays a role in regulating the MAP kinase/ERKs signaling pathway, which affects cell division, differentiation, and survival. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T598I, V599D, V599E, V599K, V599R, K600E, and A727V, and most of these mutations are clustered to two regions: the glycine-rich P loop of the N lobe and the activation segment and flanking regions.

In some embodiments, a human BRAF gene refers to the gene described by NCBI Entrez Gene ID No. 673, including mutants and variants thereof. In other embodiments, the BRAF gene is from one of the following organisms: mouse (see, e.g., NCBI Entrez Gene ID No. 109880), rat (see, e.g., NCBI Entrez Gene ID No. 114486), fish (see, e.g., NCBI Entrez Gene ID No. 403065), dog (see, e.g., NCBI Entrez Gene ID No. 475526), cattle (see, e.g., NCBI Entrez Gene ID No. 536051), horse (see, e.g., NCBI Entrez Gene ID No. 100065760), or chimpanzee (see, e.g., NCBI Entrez Gene ID No. 463781). In some embodiments, the human BRAF gene has the sequence set forth in RefSeq NM_001378474 encoding the amino acid sequence set forth in NP_001365403, the sequence set forth in RefSeq NM_001378470 encoding the amino acid sequence set forth in NP_001365399, the sequence set forth in RefSeq NM_001378475 encoding the amino acid sequence set forth in NP_001365404, the sequence set forth in RefSeq NM_001378471 encoding the amino acid sequence set forth in NP_001365400, the sequence set forth in RefSeq NM_001378468 encoding the amino acid sequence set forth in NP_001365397, the sequence set forth in RefSeq NM_001354609 encoding the amino acid sequence set forth in NP_001341538, the sequence set forth in RefSeq NM_001374258 encoding the amino acid sequence set forth in NP_001361187, the sequence set forth in RefSeq NM_001378467 encoding the amino acid sequence set forth in NP_001365396, the sequence set forth in RefSeq NM_001378472 encoding the amino acid sequence set forth in NP_001365401, the sequence set forth in RefSeq NM_001378469 encoding the amino acid sequence set forth in NP_001365398, the sequence set forth in RefSeq NM_004333 encoding the amino acid sequence set forth in NP_004324, the sequence set forth in RefSeq NM_001374244 encoding the amino acid sequence set forth in NP_00136117, or the sequence set forth in RefSeq NM_001378473 encoding the amino acid sequence set forth in NP_001365402.

### Pharmaceutical Compositions

Any of the pharmaceutical compositions disclosed herein can be formulated for use in the preparation of a medicament, and particular uses are indicated below in the context of treatment, e.g., the treatment of a subject having cancer. When employed as pharmaceuticals, any of the nucleic acids and vectors can be administered in the form of pharmaceutical compositions. Administration may be pulmonary (e.g., by inhalation or insufflation of powders or aerosols, including by nebulizer; intratracheal, intranasal, epidermal and transdermal), topical (including ophthalmic and to mucous membranes including intranasal, vaginal and rectal delivery), ocular, oral or parenteral. Parenteral administration includes intravenous, intra-arterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

In some embodiments, pharmaceutical compositions can contain, as the active ingredient, nucleic acids and vectors described herein in combination with one or more pharmaceutically acceptable carriers. The term “pharmaceutically acceptable” refers to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the pharmaceutical compositions disclosed herein, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

The nucleic acid sequences disclosed herein can be delivered to an appropriate cell of a subject, e.g. a cancer cell. This can be achieved by, for example, the use of a polymeric, biodegradable microparticle or microcapsule delivery vehicle, sized to optimize phagocytosis by phagocytic cells such as macrophages. Delivery of “naked DNA” (i.e., without a delivery vehicle) to an intramuscular, intradermal, or subcutaneous site, is another means to achieve in vivo expression. In the relevant polynucleotides (e.g., expression vectors) the nucleic acid sequence encoding the isolated nucleic acid sequence comprising a sequence encoding a CRISPR-associated endonuclease and a guide RNA can be operatively linked to a promoter or enhancer-promoter combination. Promoters and enhancers are described above.

In some embodiments, the pharmaceutical compositions can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The nucleic acids and vectors may also be applied to a surface of a device (e.g., a catheter) or contained within a pump, patch, or other drug delivery device. The nucleic acids and vectors disclosed herein can be administered alone, or in a mixture, in the presence of a pharmaceutically acceptable excipient or carrier (e.g., physiological saline). The excipient or carrier is selected on the basis of the mode and route of administration. Suitable pharmaceutical carriers, as well as pharmaceutical necessities for use in pharmaceutical formulations, are described in, e.g., Remington: The Science and Practice of Pharmacy (Adejare, ed., 23^{rd} Edition, Nov. 13, 2020) and in the USP/NF (United States Pharmacopeia and the National Formulary).

In some embodiments, the compositions can be formulated as a nanoparticle encapsulating a nucleic acid encoding a CRISPR-associated endonuclease and a guide RNA sequence complementary to an NRAS gene (or, in some embodiments, a variant NRAS gene), or vector comprising a nucleic acid encoding a CRISPR-associated endonuclease and a guide RNA sequence complementary to an NRAS gene (or, in some embodiments, a variant NRAS gene).

### Methods of Reducing Gene Expression or Activity in a Cell

In certain aspects, the disclosure relates to a method of reducing NRAS expression or activity in a cancer cell comprising introducing into the cancer cell (a) one or more nucleic acid sequence(s) encoding a guide RNA (gRNA) that is complementary to a target sequence in the NRAS gene and (b) a nucleic acid sequence encoding a Clustered Regularly Interspaced Short Palindromic Repeat (CRISPR)-associated endonuclease, whereby the gRNA hybridizes to the NRAS gene and the CRISPR-associated endonuclease cleaves the NRAS gene, and wherein NRAS expression or activity is reduced in the cancer cell relative to a cancer cell in which the one or more nucleic acid sequences encoding the gRNA and the nucleic acid sequence encoding the CRISPR-associated nuclease are not introduced.

Reducing NRAS expression in the cancer cell may comprise reducing expression of NRAS mRNA in the cancer cell, reducing expression of the NRAS protein in the cancer cell, reducing activity of the NRAS protein in the cancer, or any combination thereof. In some embodiments, introducing the one or more nucleic acid sequence(s) encoding the gRNA and the nucleic acid sequence encoding a CRISPR-associated endonuclease into the cancer cell reduces NRAS expression and/or activity in the cancer cell, but does not completely eliminate it. In other embodiments, NRAS expression and/or activity in the cancer cell are completely eliminated.

The gRNA may be complementary to a target sequence in an exon of the NRAS gene. In some embodiments, the gRNA is encoded by a single nucleic acid sequence. In other embodiments, the gRNA is encoded by two or more nucleic acid sequences. For example, in some embodiments, the gRNA is encode by a first nucleic acid sequence encoding a trans-activated small RNA (tracrRNA) and a second nucleic acid sequence encoding a CRISPR RNA (crRNA). The tracrRNA and crRNA may hybridize within the cell to form the guide RNA. Accordingly, in some embodiments, the gRNA comprises a trans-activated small RNA (tracrRNA) and a CRISPR RNA (crRNA). In some embodiments, the gRNA comprises a crRNA.

In some embodiments, the guide RNA is complementary to a variant NRAS gene that is found only in cancer cells and not in the wild-type NRAS gene in normal (i.e., non-cancerous) cells. In some embodiments, introducing the one or more nucleic acid sequence(s) encoding the gRNA and the nucleic acid sequence encoding a CRISPR-associated endonuclease into the cancer cell reduces variant NRAS expression and/or activity in the cancer cell, but does not completely eliminate it. In other embodiments, variant NRAS expression and/or activity in the cancer cell are completely eliminated.

In some embodiments, CRISPR-associated endonucleases suitable for use in reducing expression of the variant NRAS gene include, but are not limited to, a class 1 CRISPR-associated endonucleases such as, e.g., Cas7 and Cas5, along with, in some embodiments, SS (Cas11) and Cas8a1; Cas8b1; Cas8c; Cas8u2 and Cas6; Cas3″ and Cas10d; Cas SS (Cas11), Cas8e, and Cas6; Cas8f and Cas6f; Cas6f; Cas8-like (Csf1); SS (Cas11) and Cas8-like (Csf1); or SS (Cas11) and Cas10. Class 2 CRISPR-associated endonucleases include type I, type V, and type VI CRISPR-Cas systems, which have a single effector molecule. In some embodiments, CRISPR-associated endonucleases suitable for use in reducing expression of the variant NRAS gene include, but are not limited to, class 2 CRISPR-associated endonucleases such as, e.g., Cas9, Cas12a (cpf1), Cas12b1 (c2c1), Cas12a2, Cas12b2, Cas12c (c2c3), Cas12d (CasY), Cas12e (CasX), Cas12f1 (Cas14a), Cas12f2 (Cas14b), Cas12f3 (Cas14c), Cas12g, Cas12h, Cas12i, Cas12j (Casϕ), Cas12k (c2c5), Cas13a (c2c2), Cas13b1 (c2c6), Cas13b2 (c2c6), Cas13bt, Cas13c (c2c7), Cas13ct, Cas13d, Cas13X, Cas13Y, c2c4, c2c8, c2c9, and/or c2c10.

Any cell containing a variant NRAS gene may be suitable for use in the methods of reducing variant NRAS expression or activity described herein. In some embodiments, the cell is a eukaryotic cell, e.g. a mammalian cell. In some embodiments, the cell is a human cell. In some embodiments, the NRAS gene is a human NRAS gene.

In certain aspects, the disclosure also relates to a cancer cell comprising a mutated NRAS gene produced by the methods of reducing NRAS expression or activity described herein. In some embodiments, the mutated NRAS gene comprises an insertion or a deletion relative to the variant NRAS gene. In some embodiments, the insertion or deletion occurs within 1, 2, 3, 4, 5, 6, 7, 8, 9, 10, 11, 12, 13, 14, 15, 16, 17, 18, 19 or 20 nucleotide(s) of a protospacer adjacent motif sequence (PAM) in the NRAS gene.

### Methods for Treatment of Cancer

In certain aspects, the disclosure relates to a method of treating cancer in a subject comprising administering to the subject a therapeutically effective amount of a pharmaceutical composition comprising a Clustered Regularly Interspaced Short Palindromic Repeat (CRISPR)-associated endonuclease and a guide RNA that is complementary to a target domain from an NRAS gene in the subject. In certain aspects, the disclosure relates to a method of treating cancer in a subject comprising administering to the subject a therapeutically effective amount of a pharmaceutical composition comprising a Clustered Regularly Interspaced Short Palindromic Repeat (CRISPR)-associated endonuclease and a guide RNA that is complementary to a target domain from a variant NRAS gene in a cancer cell in the subject.

In certain aspects, the disclosure relates to a method of treating cancer in a subject comprising administering to the subject a therapeutically effective amount of a pharmaceutical composition comprising: (a) a nucleic acid sequence encoding a guide RNA that is complementary to a target domain from an NRAS gene in the subject; and (b) a nucleic acid sequence encoding a Clustered Regularly Interspaced Short Palindromic Repeat (CRISPR)-associated endonuclease. In some embodiments, the guide RNA is complementary to a variant NRAS gene that is found only in cancer cells and not in wild-type NRAS genes in normal (i.e., non-cancerous) cells.

In certain embodiments, the cancer is treated only with the pharmaceutical composition comprising a Clustered Regularly Interspaced Short Palindromic Repeat (CRISPR)-associated endonuclease and a guide RNA that is complementary to a target domain from an NRAS gene in the subject, or only with the pharmaceutical composition comprising: (a) a nucleic acid sequence encoding a guide RNA that is complementary to a target domain from an NRAS gene in the subject; and (b) a nucleic acid sequence encoding a Clustered Regularly Interspaced Short Palindromic Repeat (CRISPR)-associated endonuclease. In some embodiments, the guide RNA is complementary to a variant NRAS gene that is found only in cancer cells and not in the wild-type NRAS gene in normal (i.e., non-cancerous) cells. In certain embodiments, the cancer is treated with the pharmaceutical compositions as described herein and an additional agent, e.g. a chemotherapeutic agent. In certain embodiments, treatment with the chemotherapeutic agent is initiated at the same time as treatment with the pharmaceutical composition. In certain embodiments, the treatment with the chemotherapeutic agent is initiated after the treatment with the pharmaceutical composition is initiated. In certain embodiments, treatment with the chemotherapeutic agent is initiated at before the treatment with the pharmaceutical composition.

In certain embodiments, the pharmaceutical compositions of the present disclosure may be utilized for the treatment of cancer wherein the subject has failed at least one prior chemotherapeutic regimen. For example, in some embodiments, the cancer is resistant to one or more chemotherapeutic agents. Accordingly, the present disclosure provides methods of treating cancer in a subject, wherein the subject has failed at least one prior chemotherapeutic regimen for the cancer, comprising administering the pharmaceutical compositions as described herein to the subject in an amount sufficient to treat the cancer, thereby treating the cancer. The pharmaceutical compositions described herein may also be utilized for inhibiting tumor cell growth in a subject wherein the subject has failed at least one prior chemotherapeutic regimen. Accordingly, the present disclosure further provides methods of inhibiting tumor cell growth in a subject, e.g. wherein the subject has failed at least one prior chemotherapeutic regimen, comprising administering the pharmaceutical compositions described herein to the subject, such that tumor cell growth is inhibited. In certain embodiments, the subject is a mammal, e.g. a human.

For example, the pharmaceutical compositions described herein may be administered to a subject in an amount sufficient to reduce proliferation of cancer cells relative to cancer cells that are not treated with the pharmaceutical composition. The pharmaceutical composition may reduce cancer cell proliferation by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are not treated with the pharmaceutical composition.

In some embodiments, the pharmaceutical composition is administered in an amount sufficient to reduce tumor growth relative to a tumor that is not treated with the pharmaceutical composition. The pharmaceutical composition may reduce tumor growth by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are not treated with the pharmaceutical composition. In a particular embodiment, administration of the pharmaceutical composition to the subject completely inhibits tumor growth.

In one embodiment, administration of a pharmaceutical composition as described herein, achieves at least stable disease, reduces tumor size, inhibits tumor growth and/or prolongs the survival time of a tumor-bearing subject as compared to an appropriate control. Accordingly, this disclosure also relates to a method of treating tumors in a human or other animal, including a subject, who has failed at least one prior chemotherapeutic regimen, by administering to such human or animal an effective amount of a pharmaceutical composition described herein. One skilled in the art would be able, by routine experimentation with the guidance provided herein, to determine what an effective amount of the pharmaceutical composition would be for the purpose of treating malignancies including in a subject who has failed at least one prior chemotherapeutic regimen. For example, a therapeutically active amount of the pharmaceutical composition may vary according to factors such as the disease stage (e.g., stage I versus stage IV), age, sex, medical complications, and weight of the subject, and the ability of the pharmaceutical composition to elicit a desired response in the subject. The dosage regimen may be adjusted to provide the optimum therapeutic response. For example, several divided doses may be administered daily, the dose may be administered by continuous infusion, or the dose may be proportionally reduced as indicated by the exigencies of the therapeutic situation.

In certain embodiments, the methods further include a treatment regimen which includes any one of or a combination of surgery, radiation, chemotherapy, e.g., hormone therapy, antibody therapy, therapy with growth factors, cytokines, and anti-angiogenic therapy.

### Combination Therapies

In certain embodiments, the pharmaceutical compositions described herein can be used in combination therapy with at least one additional anticancer agent, e.g., a chemotherapeutic agent. Small molecule chemotherapeutic agents generally belong to various classes including, for example: 1. Topoisomerase II inhibitors (cytotoxic antibiotics), such as the anthracyclines/anthracenediones, e.g., doxorubicin, epirubicin, idarubicin and nemorubicin, the anthraquinones, e.g., mitoxantrone and losoxantrone, and the podophillotoxines, e.g., etoposide and teniposide; 2. Agents that affect microtubule formation (mitotic inhibitors), such as plant alkaloids (e.g., a compound belonging to a family of alkaline, nitrogen-containing molecules derived from plants that are biologically active and cytotoxic), e.g., taxanes, e.g., paclitaxel and docetaxel, and the vinka alkaloids, e.g., vinblastine, vincristine, and vinorelbine, and derivatives of podophyllotoxin; 3. Alkylating agents, such as nitrogen mustards, ethyleneimine compounds, alkyl sulphonates and other compounds with an alkylating action such as nitrosoureas, dacarbazine, cyclophosphamide, ifosfamide and melphalan; 4. Antimetabolites (nucleoside inhibitors), for example, folates, e.g., folic acid, fiuropyrimidines, purine or pyrimidine analogues such as 5-fluorouracil, capecitabine, gemcitabine, methotrexate, and edatrexate; 5. Topoisomerase I inhibitors, such as topotecan, irinotecan, and 9-nitrocamptothecin, camptothecin derivatives, and retinoic acid; and 6. Platinum compounds/complexes, such as cisplatin, oxaliplatin, and carboplatin. Exemplary chemotherapeutic agents for use in the methods of disclosed herein include, but are not limited to, amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-II, I0-hydroxy-7-ethyl-camptothecin (SN38), capecitabine, ftorafur, 5′deoxyflurouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973 (and analogs thereof), JM-216 (and analogs thereof), epirubicin, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, acyclovir, valacyclovir, ganciclovir, amantadine, rimantadine, lamivudine, zidovudine, bevacizumab, trastuzumab, rituximab, Pentostatin, floxuridine, fludarabine, hydroxyurea, ifosfamide, idarubicin, mesna, irinotecan, mitoxantrone, topotecan, leuprolide, megestrol, melphalan, plicamycin, mitotane, pegaspargase, pipobroman, tamoxifen, teniposide, testolactone, thiotepa, uracil mustard, vinorelbine, chlorambucil, mTor, epidermal growth factor receptor (EGFR), and fibroblast growth factors (FGF) and combinations thereof which are readily apparent to one of skill in the art based on the appropriate standard of care for a particular tumor or cancer. In a particular embodiment, the chemotherapeutic agent is selected from the group consisting of cisplatin, vinorelbine, carboplatin, and combinations thereof (e.g., cisplatin and vinorelbine; cisplatin and carboplatin; vinorelbine and carboplatin; cisplatin, vinorelbine, and carboplatin).

In some embodiments, the pharmaceutical composition is administered in an amount sufficient to reduce tumor growth relative to a tumor that is treated with the at least one chemotherapeutic agent but is not treated with the pharmaceutical composition. The pharmaceutical composition may reduce tumor growth by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to cancer cells that are treated with the at least one chemotherapeutic agent but are not treated with the pharmaceutical composition.

### EXAMPLES

TABLE 1

| SEQ | | |
|---|---|---|
| ID NO | Description | gRNA 5′-3′ |
| 1 | Q61K nPAM-Cas12a | CAGCTGTATCCAGTATGTCCA |
| 2 | Q61K MT-selective- | TACTGGATACAGCTGGAAAAG |
| | SaCas9 | |
| 3 | Non-targeting | CCCAGTCACGACGTTGTAAAA |
| | (Scramble)-Cas12a | |
| 4 | Non-selective- | TTGGACATACTGGATACAGC |
| | SpCas9 | |

### Example 1: Experimental Approach for Assessing BRAFi Resistance in NRAS-Mutant Melanoma

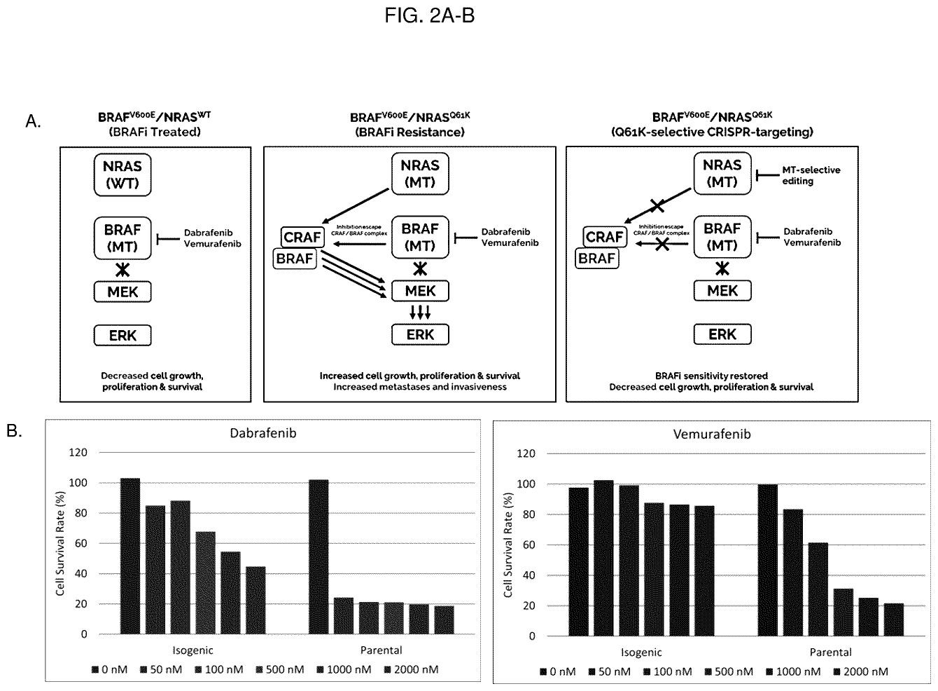

Mutations in the BRAF gene are frequently seen in patients with melanoma, with the V600E mutation being most prominent (Cheng L, Lopez-Beltran A, Massari F, et al. Molecular testing for BRAF mutations to inform melanoma treatment decisions: a move toward precision medicine. Modern Pathology 2018 31:1 2017; 31(1):24-38; Ascierto P A, Kirkwood J M, Grob J J, et al. The role of BRAF V600 mutation in melanoma. J Transl Med 2012; 10(1):1-9;). BRAF inhibitors, Dabrafenib and Vemurafenib, are a very effective first-line therapy for BRAF-mutated melanoma but resistance to this treatment is usually seen within a year, even when used in combination with MEK inhibitors (Cheng L, Lopez-Beltran A, Massari F, et al. Molecular testing for BRAF mutations to inform melanoma treatment decisions: a move toward precision medicine. Modern Pathology 2018 31:1 2017; 31(1):24-38; Lim S Y, Menzies A M, Rizos H. Mechanisms and strategies to overcome resistance to molecularly targeted therapy for melanoma. Cancer 2017; 123(S11):2118-2129; Luebker S A, Koepsell S A. Diverse Mechanisms of BRAF Inhibitor Resistance in Melanoma Identified in Clinical and Preclinical Studies. Front Oncol 2019; 9(MAR)). One mechanism of resistance to BRAFi treatment is an acquired secondary Q61K mutation in the NRAS gene. Mutant NRAS will preferentially bind with CRAF and reactivate Ras-Raf-MEK-ERK (MAPK) pathway (FIG. 2A) (Murphy B M, Terrell E M, Chirasani V R, et al. Enhanced BRAF engagement by NRAS mutants capable of promoting melanoma initiation. Nature Communications 2022 13:1 2022; 13(1):1-15). This secondary mutation not only renders treatment ineffective, but once the NRAS Q61K mutation is acquired, expression of genes involved in the epithelial-to-mesenchymal transition and paradoxically promote invasiveness and metastasis is dramatically enhanced (Chhabra G, Ahmad N. BRAF Inhibitors in Melanoma Management: When Friends Become Foes. Journal of Investigative Dermatology 2022; 142(5):1256-1259).

To assess BRAFi resistance as a result of the NRAS Q61K mutation, two human melanoma A375 cell lines were purchased from ATCC (Manassas, Virginia): A375 cell line (CRL-1619) and NRAS mutant-A375 Isogenic cell line (CRL-1619IG-2). The A375 cell line naturally harbors the BRAF V600E mutation, whereas the isogenic cell line was created at ATCC by utilizing the CRISPR/Cas9 gene editing to generate a drug resistant NRAS Q61K mutation within the A375 melanoma cell line, along with the naturally occurring BRAF V600E mutation.

To test protein bio-functionality, BRAFi treatments were performed in experimental triplicate for each condition. Cells were seeded at a 5,000 cell/well density in a 96 well opaque, white plate. Cells were dosed from 0 nM to 2000 nM concentrations for dabrafenib or vemurafenib, respectively, as determined by dose-response for each drug shown in FIG. 2B at concentrations indicated (Selleck Chemicals, Houston, Texas). Cells were then incubated at 37° C. with 5% CO₂ for 72 hours. Cellular viability was done after 72 hours using Cell Titer Glo 2.0 (Promega, Madison, Wisconsin) on a TECAN Infinite® 200 PRO (Tecan, Mannedorf, Switzerland). The NRAS mutant isogenic A375 cell line does, in fact, have increased resistance to BRAFi at increasing doses as opposed to the sensitive A375 cell line, highlighting the involvement of NRAS Q61K as a driving force for resistance to BRAFi.

### Example 2: CRISPR-Directed Mutation-Specific Targeting of NRASQ61K

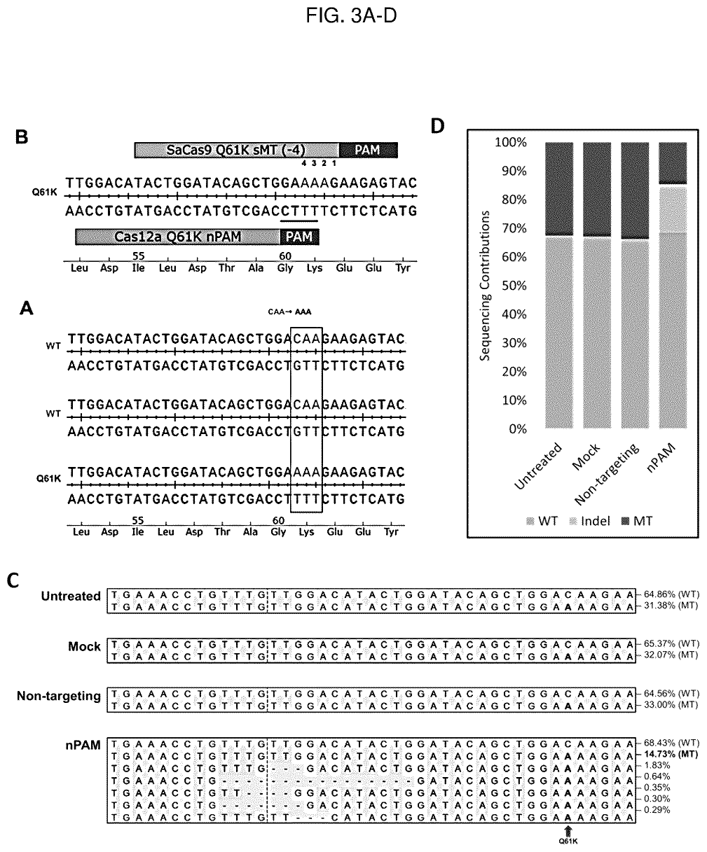

Our approach utilizes two CRISPR-directed mutation-specific targeting approaches to restore BRAFi sensitivity in BRAFV600E/NRASQ61K-mutated, BRAFi resistant A375 cells. Cas12a recognizes a PAM site of 5′-TTTN-3′, and the Q61K mutation involves a CAA to AAA nucleotide change which generates, on the complementary strand, a new 5′-TTTC-3′ PAM site from the previous Q61 5′-TGTC-3′ site (FIG. 3A).

To investigate the mutation-specific targeting selectivity of the Cas12a PAM site (nPAM) (SEQ ID NO:1) and the SaCas9 sgRNA (SEQ ID NO:2) seed region mutation (sMT) generated by the NRAS Q61K mutation, we utilized ribonucleoprotein (RNP) delivery of CRISPR molecules into isogenic A375 cells containing both the primary BRAF V600E mutation and the secondary, BRAFi resistance-inducing NRAS Q61K mutation. This cell model contains three copies of the NRAS gene, two wild-type and one Q61K-mutated, resulting in approximate allelic profile of 66% and 33%, respectively (FIG. 3B). Mismatches in PAM proximal positions 1-6 within the sgRNA are detrimental to DNA cleavage activity for SaCas9 (Zhang S, Zhang Q, Hou X-M, et al. Dynamics of Staphylococcus aureus Cas9 in DNA target Association and Dissociation. EMBO Rep 2020; 21(10): e50184; Tycko J, Barrera L A, Huston N C, et al. Pairwise library screen systematically interrogates Staphylococcus aureus Cas9 specificity in human cells. Nature Communications 2018 9:1 2018; 9(1):1-7). For the NRAS Q61K mutation, an SaCas9-targetable site is available at that location that generates an opportunistic mismatch in PAM proximal position 4 in the sgRNA, well within the critical seed region for selective targeting (FIG. 2A-B). Targeting reactions were conducted by nucleofection of preassembled RNP molecules and the editing efficiency was determined via Next Generation Sequencing (NGS) on genomic DNA (post-targeting). Sequencing showed selective Q61K targeting with the Cas12a nPAM (SEQ ID NO:1) dropped the mutated allele from approximately 33% to 15%, while the 66% wild-type contribution remained unchanged (FIG. 3C). Additionally, mock, and non-targeting RNP (scrambled) (SEQ ID NO:3) transfection complexes showed no activity, no difference from the untreated controls. These results confirm the mutation-specific cleavage selectivity of the nPAM Cas12a targeting approach.

### Example 3: CRISPR-Directed Mutation-Specific Targeting Restores BRAFi Sensitivity

Having shown that PAM creating mutations are selectively targetable with Cas12a, we next examined the selectivity of a seed region mismatch mutation that create a unique, selective cleavage opportunity for SaCas9.

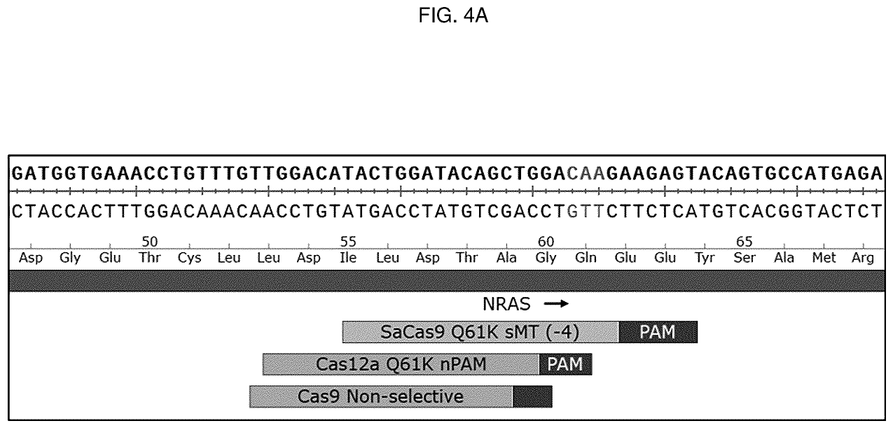

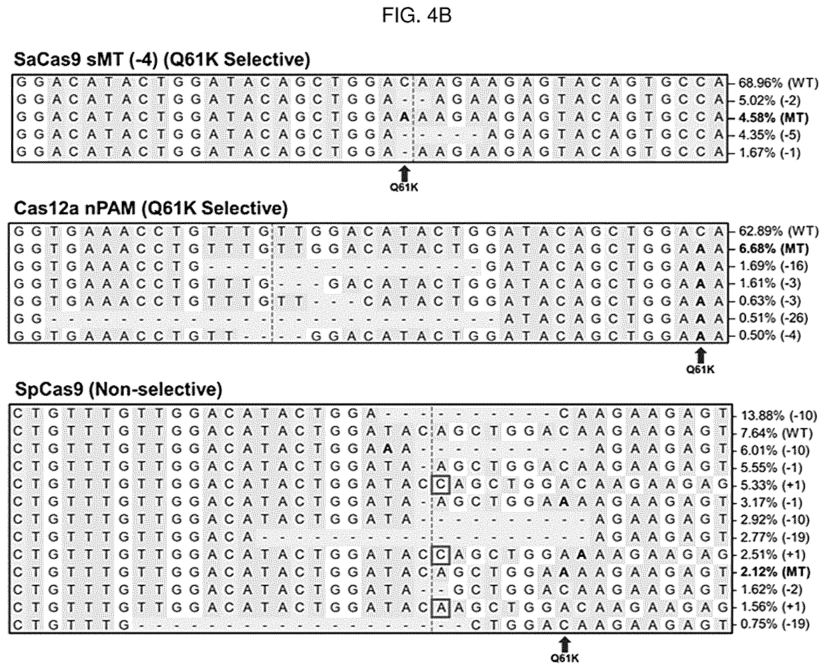

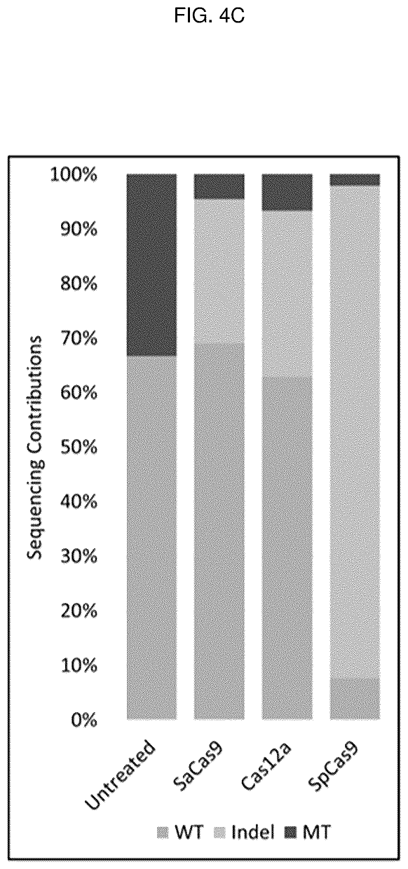

For this experiment, we used both SaCas9 sMT (SEQ ID NO:2) and Cas12a nPAM (SEQ ID NO:1) Q61K-selective RNPs and an additional non-selective (SEQ ID NO:4) SpCas9 RNP to assess the therapeutic effect of selective and non-selective disruption of the Q61/Q61K target (FIG. 4A). NGS sequencing of the targeted populations show a marked reduction in the 33% mutant allele profiles across targeting strategies (FIGS. 4B&C). For both SaCas9 sMT and Cas12a nPAM selective targeting, with only 4.58% and 6.68% of the original 33% Q61K contributions remaining, respectively. For the non-selective SpCas9 targeting (SEQ ID 4), 2.12% of the Q61K contribution remains after targeting, but at the expense of indiscriminate wild-type allele editing which also reduced the wild-type profile from 66% to 7.64%.

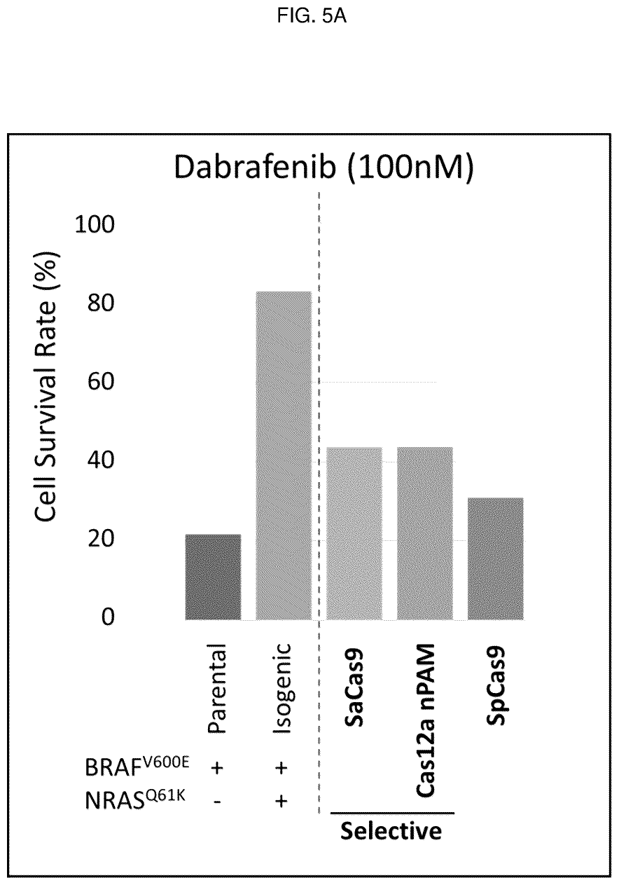

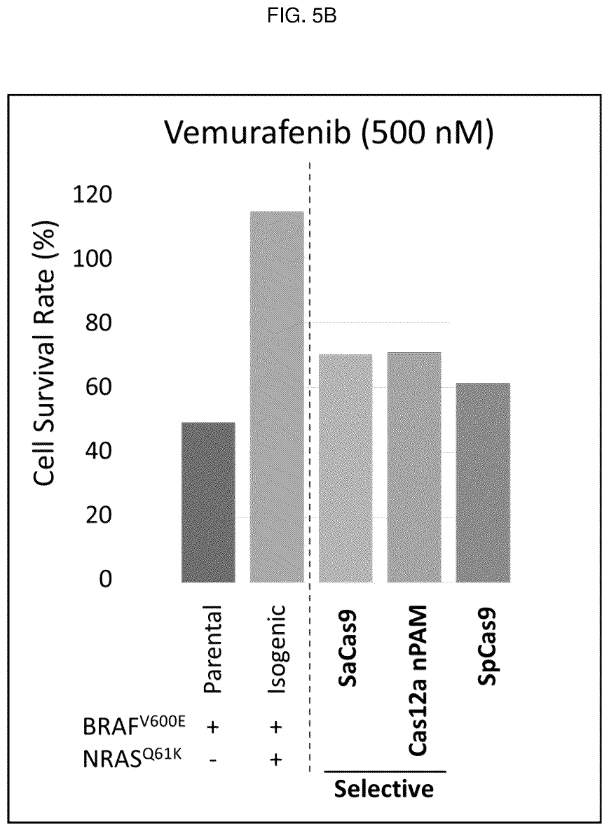

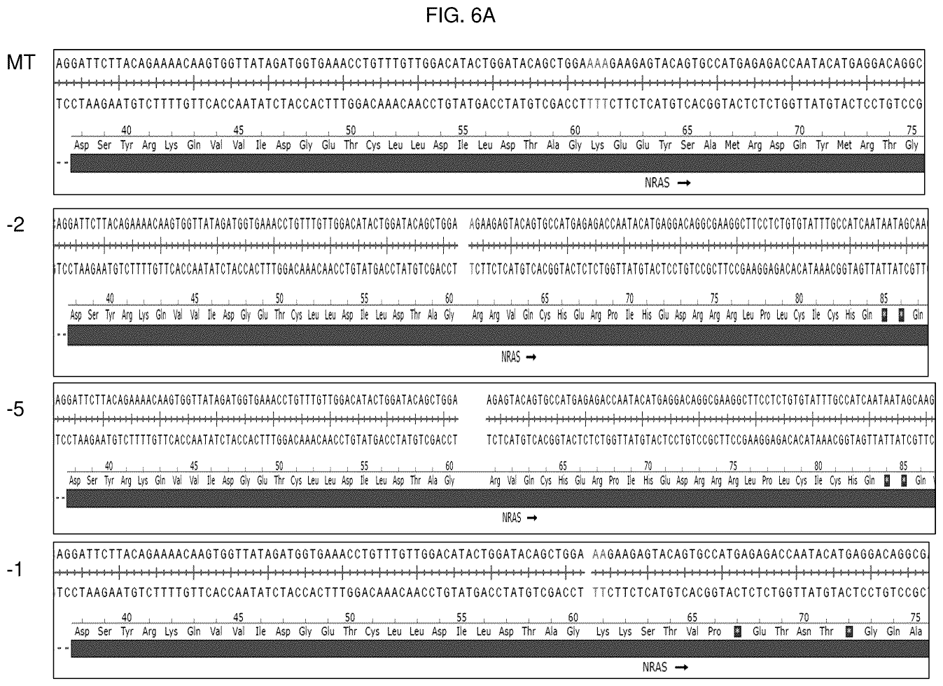

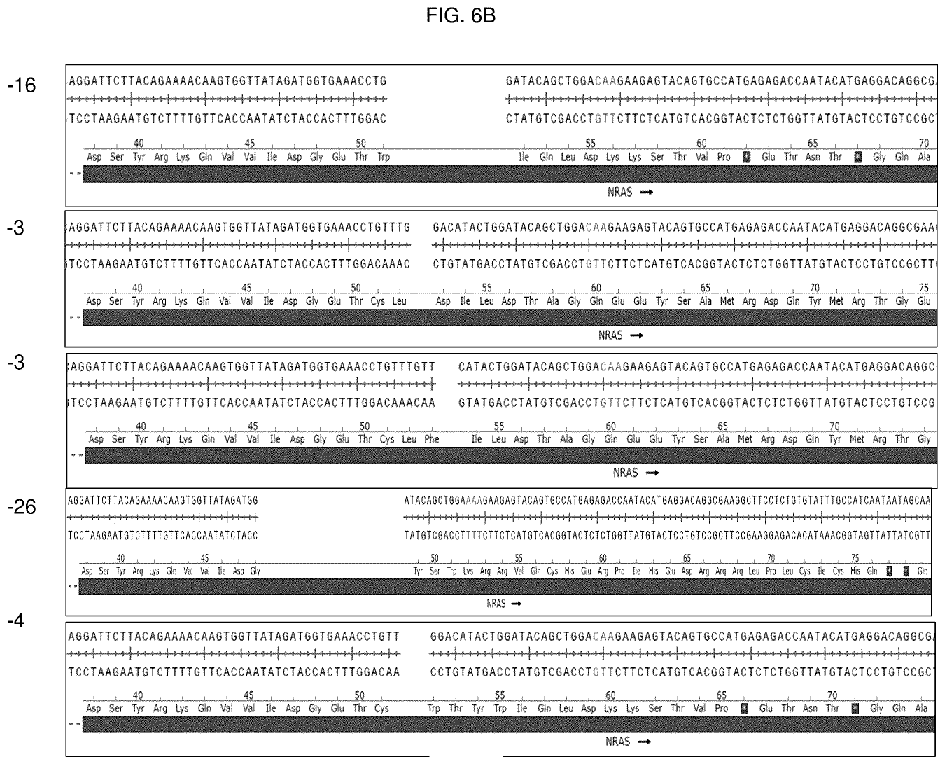

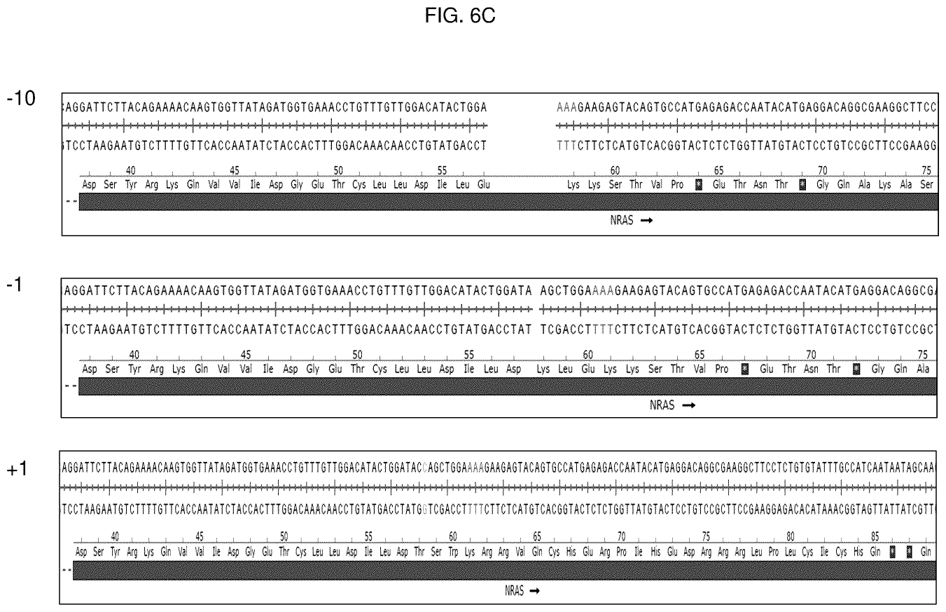

Next, we examined how these Q61K-selective targeting approaches impact functional NRAS activity with the aim of restoring sensitivity to BRAFi resistant A375 melanoma cells. We selectively targeted Q61K mutant NRAS and, after 72 hours of recovery, the targeted populations were subject to simultaneous genomic and functional analysis via NGS and BRAFi sensitivity as measured by cell viability (FIG. 5). After targeting and recovery, measurement of cell viability revealed that both selective Q61K approaches and non-selective targeting reactions successfully re-established sensitivity to previously resistance isogenic A375 cells. Cell survival for the edited populations treated with Dabrafenib (FIG. 5A) and Vemurafenib (FIG. 5B) were 44% and 70% for SaCas9, 44% and 71% for Cas12a and 31% and 61% for SpCas9, respectively. The genomic analysis additionally showed that the phenotypic response seen after BRAFi treatment was a result of the targeted editing introducing insertions and deletions (indels) which reduce the functional Q61K contribution either selectively, for Cas12a or SaCas9 targeting, or non-selectively, for SpCas9 (FIG. 6). These indels induce frameshifts within the coding sequence of the gene, leading to premature termination codons (indicated by the red squares) which typically cause nonsense-mediated decay of the altered transcript; thus impairing the expression of NRAS both at the transcriptional and protein level.

NGS analysis of Cas12a nPAM and SaCas9 sMT showed both strategies were efficient in their ability to disrupt the targeted allelic contributions and highly selective with no unintended on-site targeting occurring. We also show that that after selective (Cas12a, SaCas9) and non-selected (SpCas9) targeting, disruption of functional Q61K seen via NGS analysis reflects phenotypic changes seen in the targeted populations as re-sensitization to BRAFi. The Q61K-selective targeting with Cas12a nPAM and SaCas9 sMT showed editing efficiencies of approximately 26.3% and 28.42% of the 33% Q61K contribution, respectively, with the 66% wildtype Q61 contributions remaining unchanged. The non-selective SpCas9 targeting showed overall editing of approximately 90.2%, with 30.88% of the 33% Q61K contribution disrupted and 58.36% of the 66% wild-type contribution also having been edited. In terms of BRAFi sensitivity after targeting, the selective nPAM and sMT targeting approaches showed comparable results to the non-selective targeting with both dabrafenib and vemurafenib treatments as a direct result of the Q61K editing lowering the mutant contribution and thus re-sensitizing cells to BRAFi treatment.

### Example 4 (Prophetic): Assessment of the Reduction of NRAS Expression

To assess reduction of NRAS expression after CRISPR-directed gene editing, A375 and isogenic A375 cells are transfected with respective mutant-specific and non-selective guide RNAs. A375 and Isogenic A375 cells are split 24 hours prior to nucleofection. A 1e6 cell, 100 μl reaction is set up using SF nucleofection solution (Lonza, Morrisville, NC) with pre-complexed RNPs at 1250:250 pmol ratio (gRNA:Cas protein). AsCas12a (Ultra), SpCas9 (V3) nucleases and their respective crRNA and sgRNA are obtained from IDT (Coralville, Iowa). SaCas9 nuclease and sgRNAs are obtained from Synthego (Redwood City, CA). Nucleofection is performed using the FF-120 program on a 4D Nucleofector (Lonza, Morrisville, NC). After nucleofection, cells are plated in a 6-well plate with pre-warmed media and allowed to recover for 72 hours. Cells are harvested and total cellular protein is collected using a standard RIPA lysis buffer containing a protease inhibitor cocktail. Protein concentrations are determined using a BCA Protein Assay Kit (Pierce, Rockford, IL, USA). The samples are subjected to SDS-PAGE on a 10% polyacrylamide gel and transferred to a nitrocellulose membrane. The blot is placed in 3% BSA with TBS-T and blocked overnight on a shaker at 4° C. Primary antibody incubation is performed overnight on a shaker at 4° C. for NRAS (1:500, Abcam ab300432), and GAPDH (1:10,000, Abcam ab8245), and secondary antibody (Ab205718, Abcam) incubations are done 1 hr at room temperature at a 1:10,000 dilution. The protein bands are visualized via chemiluminescence using a SuperSignal™ West Femto Maximum Sensitivity Substrate and detected on the Azure Biosystems Gel Imager.

Alternatively, expression of NRAS after CRISPR-directed gene editing can be assessed through quantitative PCR. A375 and isogenic A375 cells are transfected with respective mutant-specific and non-selective guide RNAs. A375 and Isogenic A375 cells are split 24 hours prior to nucleofection. A 1e6 cell, 100 μl reaction is set up using SF nucleofection solution (Lonza, Morrisville, NC) with pre-complexed RNPs at 1250:250 pmol ratio (gRNA:Cas protein). AsCas12a (Ultra), SpCas9 (V3) nucleases and their respective crRNA and sgRNA are obtained from IDT (Coralville, Iowa). SaCas9 nuclease and sgRNAs are obtained from Synthego (Redwood City, CA). Nucleofection is performed using the FF-120 program on a 4D Nucleofector (Lonza, Morrisville, NC). After nucleofection, cells are plated in a 6-well plate with pre-warmed media and allowed to recover for 72 hours. Total RNA is isolated using TRIzol reagent per manufacturer's instructions and reverse transcription is conducted using the High Capacity RNA-to-cDNA kit. The cDNA is used as the template in the qPCR amplification of GAPDH and NRAS transcripts using the SsoAdvanced universal SYBR Green supermix (BioRad, Cat. 1725272). The experiment is conducted twice with all samples run in duplicate at minimum. Using the Pfaffl method, mRNA levels are calculated from Cq values and normalized to the control as indicated.

What is claimed is:

1. A method of reducing chemoresistance to one or more BRAF inhibitors in a cancer cell comprising introducing into the cancer cell (a) one or more nucleic acid sequences encoding one or more guide RNAs (gRNAs) that are complementary to one or more target sequences in a variant NRAS gene and (b) a nucleic acid sequence encoding a Clustered Regularly Interspaced Short Palindromic Repeat (CRISPR)-associated endonuclease, whereby the one or more gRNAs hybridize to the variant NRAS gene and the CRISPR-associated endonuclease cleaves the variant NRAS gene, and wherein chemoresistance to one or more BRAF inhibitors is reduced in the cancer cell relative to a cancer cell in which the one or more nucleic acid sequences encoding the one or more gRNAs and the nucleic acid sequence encoding the CRISPR-associated endonuclease are not introduced.

2. The method of claim 1, wherein the one or more gRNAs comprise a trans-activated small RNA (tracrRNA) and/or a CRISPR RNA (crRNA).

3. The method of claim 1, wherein the one or more gRNAs are one or more single guide RNAs.

4. The method of claim 1, wherein the CRISPR-associated endonuclease is a class 2 CRISPR-associated endonuclease.

5. The method of claim 4, wherein the class 2 CRISPR-associated endonuclease is Cas12a or Cas9.

6. The method of claim 1, wherein the one or more BRAF inhibitors are vemurafenib, dabrafenib, encorafenib, sorafenib, tivatinib, ARQ736, ARQ680, AZ628, CEP-32496, GDC-0879, NMS-P186, NMS-P349, NMS-P383, NMS-P396, NMS-P730, PLX3603, PLX4720, PF-04880594, PLX4734, RAF265, R04987655, SB590885, BMS908662, WYE-130600, TAK632, MLN 2480, XL281, LUT001, LUT156, LUT192, LUT195, LUT197, or a combination thereof.

7. The method of claim 1, wherein the cancer comprises a variant BRAF.

8. The method of claim 1, wherein the variant NRAS has a Q61K mutation.

9. A method of reducing variant NRAS expression or activity in a cancer cell comprising introducing into the cancer cell (a) one or more gRNAs that are complementary to one or more target sequences in the variant NRAS gene and (b) a CRISPR-associated endonuclease, whereby the one or more gRNAs hybridize to the variant NRAS gene and the CRISPR-associated endonuclease cleaves the variant NRAS gene, and wherein variant NRAS expression or activity is reduced in the cancer cell relative to a cancer cell in which the one or more gRNAs and the CRISPR-associated endonuclease are not introduced.

10. The method of claim 9, wherein the one or more gRNAs comprise a tracrRNA and/or a crRNA.

11. The method of claim 9, wherein the one or more gRNAs are one or more single guide RNAs.

12. The method of claim 9, wherein the CRISPR-associated endonuclease is a class 2 CRISPR-associated endonuclease.

13. The method of claim 12, wherein the class 2 CRISPR-associated endonuclease is Cas12a or Cas9.

14. A gRNA comprising a DNA-binding domain and a CRISPR-associated endonuclease protein-binding domain, wherein the DNA-binding domain is complementary to a target sequence in an NRAS gene.

15. A pharmaceutical composition comprising the gRNA of claim 14 and a CRISPR-associated endonuclease.

16. A ribonucleoprotein (RNP) complex comprising the gRNA of claim 14 and a CRISPR-associated endonuclease.

17. A nucleic acid sequence encoding the gRNA of claim 14.

18. A vector comprising the nucleic acid sequence of claim 17.

19. A method of treating cancer in a subject comprising administering to the subject a therapeutically effective amount of the pharmaceutical composition of claim 15.

20. The method of claim 19, wherein the cancer is resistant to one or more chemotherapeutic agents.

21. The method of claim 20, wherein the cancer is resistant to one or more BRAF inhibitors.

22. The method of claim 21, wherein the one or more BRAF inhibitors are vemurafenib, dabrafenib, encorafenib, sorafenib, tivatinib, ARQ736, ARQ680, AZ628, CEP-32496, GDC-0879, NMS-P186, NMS-P349, NMS-P383, NMS-P396, NMS-P730, PLX3603, PLX4720, PF-04880594, PLX4734, RAF265, R04987655, SB590885, BMS908662, WYE-130600, TAK632, MLN 2480, XL281, LUT001, LUT156, LUT192, LUT195, LUT197, or a combination thereof.

23. The method of claim 19, wherein the cancer comprises a variant BRAF.

24. The method of claim 19, wherein the variant NRAS has a Q61K mutation.

25. The method of claim 19, further comprising administering one or more chemotherapeutic agents to the subject.


SEQUENCE LISTING (6 sequences)

SEQ ID NO: 1 - DNA, 21 bp, synthetic construct
  source 1..21: mol_type = other DNA
cccagtcacgacgttgtaaaa

SEQ ID NO: 2 - DNA, 21 bp, synthetic construct
  source 1..21: mol_type = other DNA
tactggatacagctggaaaag

SEQ ID NO: 3 - DNA, 21 bp, synthetic construct
  source 1..21: mol_type = other DNA
cagctgtatccagtatgtcca

SEQ ID NO: 4 - DNA, 20 bp, synthetic construct
  source 1..20: mol_type = other DNA
ttggacatactggatacagc

SEQ ID NO: 5 - DNA, 4326 bp, Homo sapiens
  source 1..4326: mol_type = genomic DNA
ggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataa

SEQ ID NO: 6 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM